(19) United States
(12) Patent Application Publication
(10) Pub. No.: US 2025/0064846 A1
(43) Pub. Date: Feb. 27, 2025

(54) TREATMENT AND PREVENTION OF TRIGEMINAL NEURALGIA

(71) Applicants: THE JOHNS HOPKINS UNIVERSITY, Baltimore, MD (US); THE TRUSTEES OF COLUMBIA UNIVERSITY IN THE CITY OF NEW YORK, New York, NY (US)

(72) Inventors: Michael Lim, Baltimore, MD (US); Risheng Xu, Baltimore, MD (US); Solomon Snyder, Baltimore, MD (US); Chirag Vasavda, Baltimore, MD (US); Ryan Dhindsa, New York, NY (US)

(21) Appl. No.: 18/726,735

(22) PCT Filed: Jan. 6, 2023
(86) PCT No.: PCT/US23/60227

§ 371 Date: Jul. 3, 2024

Related U.S. Application Data
(60) Provisional application No. 63/297,547, filed on Jan. 7, 2022.

(51) Int. Cl. A61K 33/24; A61K 31/26; A61K 31/522; A61K 31/551; A61K 31/5685; A61P 25/04

(52) U.S. Cl. CPC A61K 33/24; A61K 31/26; A61K 31/522; A61K 31/551; A61K 31/5685; A61P 25/04

(57) ABSTRACT

The present disclosure provides methods of treating and preventing trigeminal nerve pain with modulators of oxidative stress (e.g., NRF2 transcription network activators and/or inhibitors of transient receptor potential ankyrin 1 (TRPA1)) and compositions thereof.

### CROSS-REFERENCE TO RELATED APPLICATIONS

This application claims the benefit of U.S. Provisional Application No. 63/297,547, filed Jan. 7, 2022, the content of which is herein incorporated by reference in its entirety.

### STATEMENT REGARDING FEDERALLY-SPONSORED RESEARCH OR DEVELOPMENT

This invention was made with government support under DA044123 and CA230285 awarded by the National Institutes of Health. The government has certain rights in the invention.

### FIELD

The present disclosure provides methods of treating and preventing trigeminal nerve pain.

### BACKGROUND

Trigeminal neuralgia is an exceedingly painful neurologic condition often characterized by sudden, short, and intense episodes of shooting, stabbing, or shock-like pain in the face. The pain can be triggered by activities of everyday life, such as eating, drinking, talking, or brushing teeth. For some patients, even a simple breeze blowing across their face can trigger excruciating pain. This pain is so debilitating that trigeminal neuralgia was historically dubbed the “suicide disease” because patients would sometimes commit suicide to end their suffering. Many more bear the pain, but endure a poor quality of life, anxiety, and depression.

Trigeminal neuralgia is thought to result from vascular compression of the trigeminal nerve, the principal sensory nerve of the face. To relieve this pressure, patients who fail conservative medical management may undergo surgical microvascular decompression in which microsurgical dissection frees the nerve from the offending artery, or the compressive vein is directly cauterized and divided. Microvascular decompression is often effective, with 61-80% of patients reporting sustained pain relief years after surgery. However, a substantial number of patients still experience persistent or recurrent pain. Moreover, approximately 25% of patients with trigeminal neuralgia do not exhibit vascular compression of the nerve from the outset. About half of these cases may be attributed to secondary causes such as multiple sclerosis or neoplasms, both of which are thought to demyelinate and injure the trigeminal nerve. In the other half, the underlying cause remains unknown.

Non-surgical medical treatments for trigeminal neuralgia often fall short, in part because the pathophysiology is incompletely understood. Currently, the only FDA-approved drug for managing trigeminal neuralgia is the anticonvulsant carbamazepine, which broadly and non-specifically inhibits neural activity. However, carbamazepine carries a significant side effect profile, including hyponatremia, leukopenia, ataxia, diplopia, and the risks of drug reaction with eosinophilia and systemic symptoms (DRESS) and Stevens-Johnson syndromes. Other drugs with fewer side effects like gabapentin, pregabalin, and antidepressants are sometimes prescribed off-label but are unfortunately less effective than carbamazepine. Up to 20-40% of trigeminal neuralgia patients are also prescribed opiates for their pain despite little evidence supporting their use, potentially worsening the socioeconomic and medical burdens of opiate overdose and addiction. Alternative treatments such as radiofrequency and/or glycerin rhizotomy or stereotactic radiosurgery use heat, chemicals, or radiation to ablate the trigeminal nerve to blunt pain. However, these procedures can leave patients with post-procedural numbness or devastating anesthesia dolorosa.

### SUMMARY

Disclosed herein are methods of treating or preventing trigeminal nerve pain in a subject comprising administering to the subject a therapeutically effective amount of at least one modulator of oxidative stress, or a composition thereof. In some embodiments, the trigeminal nerve pain comprises trigeminal neuralgia or trigeminal neuropathy.

In some embodiments, the at least one modulator of oxidative stress comprises a reactive oxygen species suppressant, an activator of anti-oxidative stress genes, or a combination thereof.

In some embodiments, the at least one modulator of oxidative stress comprises an inhibitor of transient receptor potential ankyrin 1 (TRPA1). In some embodiments, the inhibitor of TRPA1 is selected from the group consisting of ruthenium red, AM-0902, and combinations thereof.

In some embodiments, the at least one modulator of oxidative stress comprises a nuclear factor erythroid 2-related factor 2 (NRF2) transcription network activator. In some embodiments, transcription network activator is selected from the group consisting of sulforaphane, exemestane, JQ-1, and combinations thereof.

In some embodiments, the administering comprises perineural injection, systemic injection, or intranasal administration. In some embodiments, the administering comprises administration to at least one trigeminal nerve branch. In some embodiments, the administering comprises administration to sinus, inferior two-thirds of nasal cavity, or nasal septum.

Other aspects and embodiments of the disclosure will be apparent in light of the following detailed description and accompanying figures.

### BRIEF DESCRIPTION OF THE DRAWINGS

FIGS. 1A-1I show that patients and a mouse model of trigeminal neuralgia exhibit increased oxidative stress. Dot blot (FIG. 1A) and corresponding analysis (FIG. 1B) of relative 4-hydroxynonenal (4-HNE) in cerebrospinal fluid (CSF) from patients with trigeminal neuralgia (TN) was normalized to average 4-HNE in CSF of a control population (patients with Chiari malformations, normal pressure hydrocephalus, or pseudotumor cerebri). Points represent individual patients. FIG. 1C is a graph of the quantification of malondialdehyde (MDA) in CSF from patients with trigeminal neuralgia and control patients normalized to volume (μg MDA/mL CSF). Points represent individual patients. FIG. 1D is a scheme outlining the constrictive mouse model of trigeminal neuralgia and experimental timeline. One day after habituation, mice underwent constriction of the maxillary nerve or a sham surgery. From the next day onwards until Day 10, mice were scored every day for mechanical allodynia, with higher scores indicating greater allodynia. On Day 11, mice were evaluated for cold hypersensitivity by applying cold acetone to the affected vibrissal pad skin surface and measuring the time spent wiping the region in a 60 s period. Graphs of scored mechanical allodynia (FIG. 1E) and timed cold allodynia (FIG. 1F) as described in (FIG. 1D) are shown for mice that underwent constriction of the maxillary nerve or sham surgery. Points in FIG. 1E represent the mean±SEM of n=5 (sham) and 10 (ligation). Points in FIG. 1F represent individual mice. Western blots and analysis of 4-HNE (FIG. 1G) and protein carbonylation (FIG. 1H) are shown from maxillary nerves of mice that underwent constriction or sham surgery, normalized to β-actin. Lanes and points represent individual mice. FIG. 1I is a graph of the quantification of MDA from maxillary nerves of mice that underwent constriction or sham surgery normalized to protein (μg MDA/mg protein). Points represent individual mice. Median and range depicted in FIGS. 1B-1C and 1F-1I. *=P<0.05 and **=P<0.01 by two-tailed unpaired Student's t-test.

FIGS. 2A-2N show that TRPA1 is activated by reactive oxygen species and mediates trigeminal neuropathic pain. FIGS. 2A-2F are graphs of calcium imaging of HEK293 cells transiently expressing WT TRPA1 or either control vector (FIGS. 2A-2C) or mutant TRPA1 (FIGS. 2D-2F). Cells were imaged for 30 s to establish a baseline, after which vehicle was applied for 30 s. As indicated by black bars, either 100 μM iodoacetamide (FIGS. 2A and 2D), 1 mM H₂O₂ (FIGS. 2B and 2E), or 100 μM 4-HNE (FIGS. 2C and 2F) was then applied. 50 μM of the non-selective TRP channel inhibitor ruthenium red was applied for 30 s at the end of every imaging trial. FIG. 2G is a graph of calcium imaging of HEK293 cells transiently expressing WT TRPA1 in response to CSF from trigeminal neuralgia (TN) cases and controls (Ctrl). CSF was diluted into calcium imaging buffer 1:50 prior to each trial. As indicated by black bars, baseline signal was established for 30 s, after which cells were treated with vehicle. CSF was then applied for 60 s, after which cells were treated with 100 μM iodoacetamide. 50 μM of the non-selective TRP channel inhibitor ruthenium red was applied for 30 s at the end of every imaging trial. FIG. 2H is a graph of the percent of iodoacetamide-response cells activated by CSF from individual control (n=10) or trigeminal neuralgia (n=13) patients. Points represent response to CSF from individual patients. FIG. 2I is representative traces of Fluo-4 fluorescence from WT and TRPA1 trigeminal neurons in response to CSF from patients with trigeminal neuralgia. Pooled CSF was diluted into calcium imaging buffer 1:1 prior to each trial. As indicated by black bars, baseline signal was established for 30 s, after which cells were treated with vehicle. CSF was then applied for 60 s, after which cells were treated with 100 nM capsaicin. 50 mM KCl was applied at the end of every imaging trial. FIG. 2J is a graph of the percent of WT capsaicin-response neurons activated by control/trigeminal neuralgia CSF and TRPA1^{-/-} capsaicin-response neurons activated trigeminal neuralgia CSF. FIGS. 2K and 2L are graphs of scored mechanical allodynia (FIG. 2K) and timed cold allodynia (FIG. 2L) from mice following constriction of the maxillary nerve. Mice were treated with either vehicle or AM-0902 (30 mg/kg, p.o.) 30 min prior to behavior testing. Points in FIG. 2K represent the mean±SEM of n=8 (vehicle) and 10 (AM-0902). Points in FIG. 2L represent individual mice. FIGS. 2M and 2N are graphs of scored mechanical allodynia (FIG. 2M) and timed cold allodynia (FIG. 2N) from WT and TRPA1^{-/-} mice following constriction of the maxillary nerve. Points in FIG. 2M represent the mean±SEM of n=16 (WT) and 15 (TRPA1^{-/-}). Points in FIG. 2N represent individual mice. Mean±95% CI depicted with dashed lines. Median and range depicted in FIGS. 2H, 2K, 2L, and 2N. **=P<0.01 by two-tailed unpaired Student's t-test. Fraction depicted in FIG. 2J.

FIGS. 3A-3M show that NRF2 attenuates trigeminal neuropathic pain and oxidative stress. FIG. 3A is an illustration depicting mechanism of action of sulforaphane. Sulforaphane inhibits the E3-ubiquitin ligase KEAP1, which normally tags NRF2 for proteasomal degradation. Upon stabilization. NRF2 translocates to the nucleus to induce expression of antioxidant and cytoprotective genes. FIGS. 3B and 3C are graphs of scored mechanical allodynia (FIG. 3B) and timed cold allodynia (FIG. 3C) from mice that underwent constriction of the maxillary nerve or sham surgery. Mice that underwent constriction were treated with either vehicle or sulforaphane (10 mg/kg, i.p.) daily for two days before surgery and again daily just after behavior testing. Points in FIG. 3B represent the mean±SEM of n=9 (sham), 12 (vehicle), and 8 (sulforaphane). Points in FIG. 3C represent individual mice. FIGS. 3D and 3E are western blots and analysis of 4-HNE (FIG. 3D) and protein carbonylation (FIG. 3E) from maxillary nerves of mice treated with either vehicle or sulforaphane (SF) after nerve ligation, normalized to β-actin. Lanes and points represent individual mice. FIG. 3F is a graph of the quantification of MDA from maxillary nerves of mice that underwent constriction or sham surgery normalized to protein (μg MDA/mg protein). Points represent individual mice. FIG. 3G is images of NRF2 immunostaining in trigeminal ganglia from mice treated with vehicle or sulforaphane. MAP2 counterstain identifies neurons, DAPI identifies nuclei. Scale bar=30 μm. FIGS. 3H-3J are graphs of scored mechanical allodynia (FIGS. 3H and 3I) and timed cold allodynia (FIG. 3J) from WT and NRF2^{-/-} mice that underwent constriction of the maxillary nerve. Mice in FIG. 3H were not treated, whereas mice in FIGS. 3I-3J were treated with either vehicle or AM-0902 (30 mg/kg, p.o.) 30 min prior to behavior testing. Points in FIG. 3H represent the mean±SEM of n=10 (WT) and 10 (NRF2^{-/-}). Points in FIGS. 3I-3J represent individual mice. FIG. 3K is an illustration depicting mechanism of action of tamoxifen. Tamoxifen permits Cre recombinase to translocate to the nucleus, where it then targets and excises floxed exons of Keap1. Loss of Keap1 allows NRF2 to accumulate. NRF2 then translocates to the nucleus to induce expression of antioxidant and cytoprotective genes. FIGS. 3L and 3M are graphs of scored mechanical allodynia (FIG. 3L) and timed cold allodynia (FIG. 3M) from mice that underwent constriction of the maxillary nerve. Keap1(f/f) mice harbor floxed Keap1 alleles, whereas Keap1(f/f)/CMV-CreER mice also harbor a tamoxifen-inducible Cre. Both Keap1(f/f) and Keap1(f/f)/CMV-CreER were injected with tamoxifen, and behavioral tests were only performed 7 or more days after the final tamoxifen injection. Points in FIG. 3L represent the mean t SEM of n=5 (Keap1(f/f)) and 6 (Keap1(f/f)/CMV-CreER). Points in FIG. 3M represent individual mice. Median and range depicted in FIGS. 3C-3F, 3I-3J, and 3M. *=P<0.05 and **=P<0.01 by two-tailed unpaired Student's t-test.

FIGS. 4A-4M show that drug repositioning identified NRF2 network modulators as potential treatments for trigeminal neuropathic pain. FIGS. 4A and 4B show the top twenty compounds with greatest connectivity scores predicted to mimic Nfe2l2-derived and Keap1-derived transcriptome signatures. FIG. 4C is a plot of connectivity scores of molecules in Nfe2l2-derived query against the Keap1-derived query. Points represent individual molecules. JQ-1 and exemestane are emphasized, with their molecular structures to the right. FIG. 4D is a graph of firefly luciferase activity after 6 hrs exposure to varying concentrations of sulforaphane, exemestane, or JQ-1 relative to vehicle treatment, normalized to total protein. Luciferase expression is controlled by a promoter that contains several NRF2 binding sites. n=2 independent experiments in triplicate. FIG. 4E is a volcano plot of transcriptome sequencing of primary human dermal fibroblasts after 48 hours of treatment with vehicle or 0.25 μM JQ-1. Points represent individual genes. Black points indicate significantly downregulated genes (FDR<0.5, log₂ (fold change)≤−1), whereas pink points indicate significantly upregulated genes (FDR<0.5, log₂(fold change)≥1). Gray points are not differentially expressed between treatments. Notable NRF2 target genes are labeled. FIG. 4F is gene ontology analysis of molecular pathways upregulated in transcriptome sequencing in FIG. 4E. FIGS. 4G-4I are graphs of scored mechanical allodynia (FIGS. 4G and 4I) and timed cold allodynia (FIGS. 4H and 4J) from mice that underwent constriction of the maxillary nerve or sham surgery. Mice that underwent constriction were treated with either vehicle, exemestane (10 mg/kg, i.p.), or JQ-1 (40 mg/kg, i.p.) daily for two days before surgery and again daily just after behavior testing. Points in FIG. 4G represent the mean±SEM of n=4 (sham), 7 (vehicle), and 8 (exemestane). Points in FIG. 4I represent the mean±SEM of n=4 (sham), 8 (vehicle), and 4 (JQ-1). Points in FIGS. 4H and 4J represent individual mice. FIG. 4K is an image of NRF2 immunostaining in trigeminal ganglia from mice treated with vehicle or exemestane. MAP2 counterstain identifies neurons, DAPI identifies nuclei. Scale bar=30 μm. FIGS. 4L and 4M are graphs of scored mechanical allodynia (FIG. 4L) and timed cold allodynia (FIG. 4M) from mice that underwent constriction of the maxillary nerve after a single, local treatment of vehicle or exemestane (25 μg/site) to the maxillary nerve. Points in FIG. 4L represent the mean±SEM of n=5 (vehicle) and 10 (exemestane). Points in FIG. 4M represent individual mice. Mean±SEM depicted in FIG. 4D. Median and range depicted in FIGS. 4H, 4J, and 4M. *=P<0.05 and **=P<0.01 by two-tailed unpaired Student's t-test.

FIGS. 5A-5D show that CSF reactive oxygen species do not correlate CSF hemoglobin or surgery. FIGS. 5A and 5B are graphs showing the relationship between cerebrospinal fluid (CSF) hemoglobin and CSF 4-hydroxynonenal (FIG. 5A; 4-HNE) and CSF malondialdehyde (FIG. 5B; MDA). Points represent individual patients. FIG. 5C is a graph of the comparison of relative 4-HNE in CSF from patients with trigeminal neuralgia (TN) normalized to average 4-HNE in CSF from patients who underwent posterior fossa craniectomies (Craniectomy Ctrls). Points represent individual patients. FIG. 5D is a graph of the comparison of normalized MDA (μg MDA/mL CSF) in CSF from patients with trigeminal neuralgia (TN) and CSF from patients who underwent posterior fossa craniectomies (Craniectomy Ctrls). Points represent individual patients. Mean depicted in FIGS. 5C and 5D, respectively. **=P<0.01 by two-tailed unpaired Student's t-test.

FIGS. 6A-6D are images myelin basic protein immunostaining of maxillary nerves. Myelin basic protein (MBP) immunostaining of trigeminal maxillary nerves is shown from mice that underwent constriction of the maxillary nerve or a sham surgery (FIG. 6A) or underwent surgery and were treated with either vehicle or sulforaphane (FIG. 6B; 10 mg/kg, i.p.), exemestane (FIG. 6C; 10 mg/kg, i.p.), or JQ-1 (FIG. 6D; 40 mg/kg, i.p.). Scale bar=30 μm.

FIGS. 7A and 7B show the expression of WT and targeted TRPA1 cysteine/lysine mutants. FIG. 7A is a representative western blot for WT and targeted TRPA1 cysteine/lysine mutants and β-actin; top, shorter exposure, middle, longer exposure. FIG. 7B is quantification of relative expression of each TRPA1 construct relative to WT, normalized to β-actin (FIG. 7A, bottom). Points represent normalized values from n=3 independent experiments.

FIGS. 8A-8P are graphs showing TRPA1 selectively responds to CSF from patients with trigeminal neuralgia. Shown are calcium traces from HEK293 cells transiently expressing WT TRPA1 in response to CSF from control (FIGS. 8A-8H; Ctrl) and trigeminal neuralgia (FIGS. 8I-8P; TN) patients. CSF was diluted into calcium imaging buffer 1:50 prior to each trial. As indicated by black bars, baseline signal was established for 30 s, after which cells were treated with vehicle. CSF was then applied for 60 s, after which cells were treated with 100 μM iodoacetamide. 50 μM of the non-selective TRP channel inhibitor ruthenium red was applied for 30 s at the end of every imaging trial. Mean±95% CI depicted with dashed lines.

FIG. 9 is traces of Fluo-4 fluorescence from Schwann cells in response to CSF trigeminal neuralgia (TN) patients. CSF was diluted into calcium imaging buffer 1:50 prior to each trial. As indicated by black bars, baseline signal was established for 30 s, after which cells were treated with vehicle. CSF was then applied for 120 s. Mean±95% CI depicted with dashed lines from n=16 cells. Inlet plots data along a narrower range to permit better inspection of Fluo-4 fluorescence.

FIGS. 10A-10D show that TRPA1 null males and females both exhibit less mechanical and thermal pain. Scored mechanical allodynia (FIGS. 10A and IOC) and timed cold allodynia (FIGS. 10B and 10D) from male WT and TRPA1−/− mice (FIGS. 10A-10B) and female WT and TRPA1−/− mice (FIGS. 10C-10D) following constriction of the maxillary nerve are shown. Points in FIG. 10A scored mechanical allodynia represent the mean±SEM of n=8 (WT) and 7 (TRPA1−/−). Points in FIG. 10C scored mechanical allodynia represent the mean±SEM of n=8 (WT) and 8 (TRPA1−/−). Points in timed cold allodynia represent individual mice. Mean±95% CI depicted with dashed lines in scored mechanical allodynia graphs. Median and range depicted in timed cold allodynia. **=P<0.01 by two-tailed unpaired Student's t-test.

FIGS. 11A and 11B show sulforaphane comparatively lowered mechanical or cold allodynia in TRPA1−/− mice. Scored mechanical allodynia (FIG. 11A) and timed cold allodynia (FIG. 11B) from WT and TRPA1^{-/-} mice that underwent constriction of the maxillary nerve are shown. Mice were treated with either vehicle or sulforaphane (10 mg/kg, i.p.) daily for two days before surgery and again daily just after behavior testing. Points in FIG. 11A represent the mean±SEM of n=5 (WT, vehicle), 5 (TRPA1^{-/-}, vehicle), 6 (WT, sulforaphane), and 5 (TRPA1^{-/-}, sulforaphane). Points in FIG. 11B represent individual mice. Median and range depicted. **=P<0.01 and ns=P>0.05 by two-tailed unpaired Student's t-test.

FIGS. 12A-12F show genetic ablation of Keap1. The Mus musculus Keap1 gene consists of 6 exons (FIGS. 12A and 12B). To generate a mouse line in which Keap1 can be targeted for deletion, exons 2-3 were specifically flanked by two LoxP sites to facilitate excision by Cre recombinase (Keap1(f/f)). In order to globally and inducibly delete Keap1, Keap1(f/f) was crossed to a mouse harboring a tamoxifen-inducible Cre recombinase (CMV-Cre^{ERT2}) to generate Keap1(f/f)/CMV-CreER. To induce excision of Keap1 in fibroblasts, Keap1^{f/f}/CMV-CreER cells were treated with 1 μM 4-hydroxytamoxifen (4-OHT) on one day and then again 2 days later (FIG. 12C, top). Keap1^{f/f}/CMV-CreER cells were treated with vehicle and served as controls. Keap1^{f/f}/CMV-CreER mice were injected intraperitoneally with 75 mg/kg tamoxifen once every 24 hrs over 5 consecutive days (FIG. 12C, bottom). Keap1^{f/f} (Cre-negative) mice were similarly injected with tamoxifen and served as controls. FIG. 12D shows representative PCR results of DNA for exons 2-3 of Keap1 from Keap1f/f/CMV-CreER fibroblasts treated with either vehicle or 4-hydroxytamoxifen. FIGS. 12E and 12F are graphs of quantitative PCR analysis of Keap1 (FIG. 12B) and Nqo1 (FIG. 12C) mRNA normalized to Actb from Keap1(f/f) and Keap1f/f/CMV-CreER mice after treatment with tamoxifen. Points represent individual mice; median and range depicted. **=P<0.01 by two-tailed unpaired Student's t-test.

FIGS. 13A-13F show neither letrozole nor (−)-JQ-1 replicated the analgesic effects of exemestane or (+)-JQ-1. Molecular structures of exemestane and letrozole (FIG. 13A) and (+)-JQ-1 and (−)-JQ-1 (FIG. 13B). Scored mechanical allodynia (FIGS. 13C and 13E) and timed cold allodynia (FIGS. 13D and 13F) from mice that underwent constriction of the maxillary nerve or sham surgery are shown. Mice that underwent constriction were treated with either vehicle, exemestane (10 mg/kg, i.p.), letrozole (10 mg/kg, i.p.), (+)-JQ-1 (40 mg/kg, i.p.), or (−)-JQ-1 (40 mg/kg, i.p.) daily for two days before surgery and again daily just after behavior testing. Points in FIG. 13C represent the mean±SEM of n=4 (sham), 4 (vehicle), 6 (exemestane), and 6 (letrozole). Points in FIG. 13E represent the mean±SEM of n=4 (sham), 4 (vehicle), 7 ((+)-JQ-1), and 7 ((−)-JQ-1). Points in FIGS. 13D and 13F represent individual mice. Median and range depicted. **=P<0.01 and ns=P>0.05 by two-tailed unpaired Student's t-test.

FIGS. 14A-14C are graphs of calcium imaging of HEK293 cells transiently expressing WT TRPA1. Cells were imaged for 30 s to establish a baseline, after which 4-HNE was applied for 30 s. As indicated by black bars, either 1 μM exemestane (FIG. 14A), 100 μM JQ-1 (FIG. 14B), or 10 μM sulforaphane (FIG. 14C) was then applied. 50 μM of the non-selective TRP channel inhibitor ruthenium red was applied for 30 s at the end of every imaging trial. Mean±95% CI depicted with dashed lines.

FIGS. 15A-15H are western blots for TRPA1 (FIGS. 15A, 15C, 15E, and 15G) and graphs of relative TRPA1 expression (FIGS. 15B, 15D, 15F and 15H) from maxillary nerves of mice following constriction compared to sham surgery (FIG. 15A-15B) or treatment with sulforaphane (FIG. 15C-15D), exemestane (FIG. 15E-15F), or JQ-1 (FIG. 15G-15H), normalized to β-actin. Lanes and points represent individual mice. Median and range depicted in graphs of relative TRPA1 expression. ns=P>0.05 by two-tailed unpaired Student's t-test.

FIGS. 16A-16D show exemestane and JQ-1 limited protein carbonylation and 4-HNE. FIGS. 16A and 16B are western blots and analysis of 4-HNE (FIG. 16A) and protein carbonylation (FIG. 16B) from maxillary nerves of mice treated with either vehicle or exemestane after nerve ligation, normalized to β-actin. Lanes and points represent individual mice. FIGS. 16C and 16D are western blots and analysis of 4-HNE (FIG. 16C) and protein carbonylation (FIG. 16D) from maxillary nerves of mice treated with either vehicle or JQ-1 after nerve ligation, normalized to β-actin. Lanes and points represent individual mice. Median and range depicted. *=P<0.05, **=P<0.01, and ns=P>0.05 by two-tailed unpaired Student's t-test.

FIGS. 17A-17J show differential upregulation of NRF2 target genes after treatment with exemestane and JQ-1. Quantitative PCR analysis of Ngo1, Hmox1 Txnrd1, Prdx1, and Gclc, as indicated, normalized to Actb from maxillary nerves of mice after 11 days of treatment with either exemestane (FIGS. 17A-17E; 10 mg/kg, i.p.) or JQ-1 (FIGS. 17F-17J; 40 mg/kg, i.p.) compared to vehicle is shown. Points represent individual mice. Median and range depicted. *=P<0.05. **=P<0.01, and ns=P>0.05 by two-tailed unpaired Student's t-test.

FIGS. 18A and 18B are graphs of scored mechanical allodynia (FIG. 18A) and timed cold allodynia (FIG. 18B) from mice that underwent constriction of the maxillary nerve. Mice were treated with either exemestane (10 mg/kg, i.p.), the exemestane vehicle, ascorbate (100 mg/kg, i.p.), or the ascorbate vehicle daily for two days before surgery and again daily just after behavior testing. Points in FIG. 18A represent the mean±SEM of n=5 (exemestane), 4 (exemestane vehicle), 5 (ascorbate), and 4 (ascorbate vehicle). Points in FIG. 18B represent individual mice with median and range depicted. **=P<0.01 and ns=P>0.05 by two-tailed unpaired Student's t-test. FIGS. 18C and 18D are immunoblots and quantification of 4-HNE (FIG. 18C) and protein carbonylation (FIG. 18D) from maxillary nerves of mice treated with either vehicle or ascorbate after nerve constriction, normalized to β-actin. Lanes and points represent individual mice. In FIGS. 14C-14D median and range depicted. **=P<0.01 and ns=P>0.05 by two-tailed unpaired Student's t-test.

FIGS. 19A-19D are graphs of calcium imaging of HEK293 cells transiently expressing WT TRPA1. Cells were imaged for 30 s to establish a baseline, after which vehicle was applied for 30 s. As indicated by black bars, either 100 μM exemestane (FIG. 19A), 1 μM exemestane (FIG. 19B), 100 μM JQ-1 (FIG. 19C), or 10 μM sulforaphane (FIG. 19D; SF) was then applied. 4-HNE was applied afterwards to identify TRPA1-expressing cells. 50 μM of the non-selective TRP channel inhibitor ruthenium red was applied for 30 s at the end of every imaging trial. Mean±95% CI depicted with dashed lines.

FIG. 20 is representative traces of Fluo-4 fluorescence from independent WT trigeminal neurons on one cover slip in response to CSF from patients with trigeminal neuralgia. Pooled CSF was diluted into calcium imaging buffer 1:1 prior to each trial. As indicated by black bars, baseline signal was established for 30 s, after which cells were treated with vehicle. CSF was then applied for 60 s, after which cells were treated with 100 nM capsaicin. 50 mM KCl was applied at the end of every imaging trial.

FIGS. 21A-21D show that JQ-1's mechanism of action differs from sulforaphane and exemestane. To determine a ratio of KFAP1 to NRF2 cDNA that best mimics baseline NRF2 activity, luciferase activity was measured 48 h after transfection with varying ratios of KEAP1 to NRF2 cDNA, normalized to total protein (FIG. 21A). Luciferase expression is controlled by a promoter that contains several NRF2 binding sites and is thus a measure of NRF2 activity. A 2.5 μg KFAP1 to 1 μg NRF2 cDNA ratio returned luciferase activity to baseline and was therefore employed for subsequent biochemical studies evaluating NRF2 stability and nuclear translocation. FIGS. 21B and 21C show the quantification (FIG. 21B) and immunoblots (FIG. 21C) of myc-NRF2, GAPDH, and H2B in whole cell lysates and cytoplasmic and nuclear subcellular fractions of HEK-293 cells overexpressing FLAG-KEAP1 and myc-NRF2, treated with vehicle, 10 μM MG-132, 10 μM sulforaphane (SF), 10 μM exemestane, or 10 μM JQ-1 for 6 hours. Data are expressed as a normalized ratio of nuclear myc-NRF2 compared to the vehicle-treated condition. FIG. 21D is immunoblots of lysates (input) and myc immunoprecipitate (IP) from HEK-293 cells overexpressing FLAG-KEAP1, myc-NRF2, and HA-ubiquitin. Data are either representative of or quantified from n=3 independent experiments except in FIG. 21A, where n=1 in triplicate. In FIG. 21A, mean f SEM depicted. In FIG. 21B, median and range depicted. *=P<0.05 and ns=P>0.05 by one-way ANOVA followed by post-hoc Dunnett's test.

FIGS. 22A-22D are graphs showing 4-HNE and H₂O₂ activate NRF2 at lower concentrations than TRPA1. NRF2-dependent luciferase activity (FIGS. 22A and 22C) and concentration—Ca²⁺ response curves (FIGS. 22B and 22D) after varying concentrations of 4-HNE (FIGS. 22A-22B) and H₂O₂ (FIGS. 22C-22D). Cells in FIGS. 22A and 22C were treated for 6 hours to allow for transcription and translation of luciferase. HEK-293 cells in FIGS. 22B and 22D were transfected with either WT TRPA1 cDNA or the empty vector. Data are a representative experiment of 2-4 independent experiments performed in triplicate, depicted as mean±SEM.

FIGS. 23A-23F are graphs of scored mechanical allodynia (FIGS. 23A, 23C, and 23E) and timed cold allodynia (FIGS. 23B, 23D, and 23F) from WT and NRF2^{-/-} mice that underwent constriction of the maxillary nerve or sham surgery. Mice that underwent constriction were treated with either vehicle, sulforaphane (10 mg/kg, i.p.; FIGS. 23A-23B), exemestane (10 mg/kg, i.p.; FIGS. 23C-23D), or JQ-1 (40 mg/kg, i.p.; FIGS. 23E-23F) daily for two days before surgery and again daily just after behavior testing. Mice treated with sulforaphane, exemestane, or JQ-1 were all compared to the same vehicle-treated mice of the matching genotype regardless of drug treatment (uniformly depicted with reduced opacity in each plot). Points in FIGS. 23A-23F representing vehicle mice are measurements from n=4 (WT) or n=4 (NRF2^{-/-}). Points in FIGS. 23A, 23C, and 23E represent the mean±SEM of vehicle-treated mice and: in FIG. 23A, n=5 (WT) and 4 (NRF2^{-/-}); in FIG. 23C, n=4 (WT) and 5 (NRF2^{-/-}); in FIG. 23E, n=6 (WT) and 5 (NRF2^{-/-}). Points in FIGS. 23B, 23D, and 23F represent individual mice. Median and range depicted. **=P<0.01, *=P<0.05, and ns=P>0.05 by two-tailed unpaired Student's t-test.

FIGS. 24A and 24B are compound connectivity scores, above CMAP's recommended cutoff of +90, for Nfe2l2-derived (FIG. 24A) and Keap1-derived (FIG. 24B) transcriptome signatures.

### DETAILED DESCRIPTION

The present disclosure provides methods of treating or preventing trigeminal nerve pain or trigeminal nerve injury in a subject comprising administering to the subject a therapeutically effective amount of at least one modulator of oxidative stress, or a composition thereof.

By leveraging a combined clinical, molecular, and computational approach, the NRF2 transcriptional network was identified as a therapeutic target for trigeminal neuralgia. Even through divergent mechanisms, inducing the NRF2 network was analgesic in the constrictive mouse model of trigeminal neuralgia. In contrast to current pharmacologic agents that mask pain by blunting nerve firing, increasing the NRF2 transcriptional network improves pain through neuroprotection.

Exemestane and JQ-1 were identified as two NRF2 network modulators for treating trigeminal neuropathic pain. Exemestane induced the NRF2 network through NRF2 itself, whereas JQ-1 recruited the network independent of NRF2. Though exemestane is best known as an aromatase inhibitor, other structurally dissimilar aromatase inhibitors do not induce Nqo1, suggesting that exemestane's NRF2 activity is not tied to inhibitory aromatase activity. Exemestane exerted a powerful analgesic effect that persists over the course of days to weeks.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the term “perineural” refers to administration directly to, proximal to, or within the tissues surrounding at least one nerve of a subject. In some embodiments, perineural administration may be a nerve block.

As used herein, the terms “providing”, “administering,” “introducing,” are used interchangeably herein and refer to the placement of the at least one modulator of oxidative stress or compositions of the disclosure into a subject by a method or route which results in at least partial localization to a desired site. The modulators of oxidative stress or compositions can be administered by any appropriate route which results in delivery to a desired location in the subject.

As used herein, the terms “effective amount” or “therapeutically effective amount,” refer to a sufficient amount of the modulator of oxidative stress or a composition or a combination of compositions thereof being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of a composition as described herein required to provide a clinically significant decrease in disease symptoms.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, “treat,” “treating” and the like means a slowing, stopping, or reversing of progression of a disease or disorder when provided a modulator of oxidative stress or composition described herein to an appropriate subject. The term also includes a reversing of the progression of such a disease or disorder to a point of eliminating or greatly reducing the disease. As such, “treating” means an application or administration of the modulator(s) of oxidative stress or compositions described herein to a subject, where the subject has a disease or a symptom of a disease, where the purpose is to cure, heal, alleviate, relieve, alter, remedy, ameliorate, improve, or affect the disease or symptoms of the disease.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. METHODS OF TREATMENT

The present disclosure provides methods for treating or preventing trigeminal nerve pain in a subject. The trigeminal nerve pain may be an acute or chronic pain. Some forms of acute pain can develop into chronic pain through a progressive and complex process. In some embodiments, the methods may prevent the transition of acute trigeminal nerve pain from developing into a chronic pain state. The trigeminal nerve pain may be procedural related pain. In some examples, the procedural-related pain is pain arising from dental, medical, surgical, or cosmetic procedures.

Chronic, acute, or procedural pain associated with the trigeminal nerve system is experienced in many syndromes and diseases including, but not limited to, trigeminal neuralgia, trigeminal neuropathy, facial pain, anesthesia dolorosa, post-herpetic neuralgia, cancer of the head and neck, migraine headaches, other types of headaches, TMJ, injuries to the face and/or head, injuries or infections of the teeth, common dental procedures, and facial surgeries such as cosmetic plastic surgery.

Due to the relative overlap of trigeminal nerve disorder symptoms and lack of known etiology, trigeminal nerve pain diagnoses can vary by physician. The methods described herein may be used to treat any cause of pain associated with the trigeminal nerve, including, for example trigeminal neuralgia (type 1 or type 2) and trigeminal neuropathy.

Type 1 trigeminal neuralgia, also known as classical trigeminal neuralgia, includes cases that develop idiopathically or secondary to neurovascular compression, but largely the mechanisms underlying trigeminal neuralgia are not entirely understood. The neurovascular compression causes a wearing away of or damage to the protective coating around the trigeminal nerve. Type 1 trigeminal neuralgia is defined clinically by attacks of usually intense, sharp, superficial, or stabbing pain in the distribution of one or more branches of the trigeminal nerve. The pain of trigeminal neuralgia tends to occur in paroxysms and is maximal at or near onset. Facial muscle spasms may be seen with severe pain. The pain can be triggered by activities of everyday life, such as eating, drinking, talking, or brushing teeth. For some patients, even a simple breeze blowing across their face can trigger excruciating pain. The pain can be so intensely debilitating that trigeminal neuralgia was historically dubbed the “suicide disease” because patients would sometimes commit suicide to end their suffering. Many more bear the pain, but endure a poor quality of life, characterized by anxiety and depression in addition to painful.

Type 2 trigeminal neuralgia, also known as atypical trigeminal neuralgia, can have a wide range of symptoms and the pain can fluctuate in intensity from mild aching to a crushing or burning sensation, and also to the extreme pain experienced with the more common trigeminal neuralgia. Symptoms of type 2 trigeminal neuralgia often overlap with other disorders, including migraine, atypical odontalgia, and post herpetic neuralgia. Patients with type 2 trigeminal neuralgia typically experience a persistent dull ache or burning sensation in at least one part of the face. However, episodes of sharp pain can complicate type 2 trigeminal neuralgia. Unlike type 1 trigeminal neuralgia, there is often not a specific trigger point for the pain and it can grow worse over time. Type 2 trigeminal neuralgia can be idiopathic, due to compression of the trigeminal nerve, or can occur due to a known underlying cause such as a tumor or multiple sclerosis.

Painful trigeminal neuropathy is caused by structural abnormalities or neural damage rather than vascular compression. Painful trigeminal neuropathy affects one or more branches of the trigeminal nerve, most often the second (V2, maxillary) or third (V3, mandibular) division. Common causes of painful trigeminal neuropathy include multiple sclerosis, tumors, acute herpes zoster, post-chemotherapy neuritis, post-radiation therapy neuritis, and space occupying abnormalities. The pain associated with painful trigeminal neuropathy is highly variable in quality and intensity. Painful trigeminal neuropathy is characterized by continuous or near-continuous facial pain often described subjectively as burning, squeezing, shock-like, or likened to pins and needles.

The methods may decrease the severity of the pain experienced in the subject, may decrease the frequency of the pain attacks, may decrease the length of the pain attacks, may decrease the susceptibility of the subject to triggers of pain, may increase the quality of life of the patient, or a combination thereof. In some embodiments, the nerve pain is trigeminal neuralgia. In some embodiments, the nerve pain is trigeminal neuropathy.

The methods described herein comprise administering to the subject a therapeutically effective amount of at least one modulator of oxidative stress, or a composition thereof. Modulators of oxidative stress include, without limitation, reactive oxygen species suppressants and activators of anti-oxidative stress genes.

In some embodiments, the at least one modulator of oxidative stress comprises an inhibitor of transient receptor potential ankyrin 1 (TRPA1). TRPA1 inhibitors are known in the art and include, without limitation, ruthenium red, AM-0902 (CAS No. 1883711-97-4), HC-030031 (CAS No. 349085-38-7), xanthine derivatives (e.g., Chembridge-5861528 (CAS No. 332117-28-9)), A-967079 (CAS No. 1170613-55-4), and trichloro(sulfanyl)ethyl benzamides. In some embodiments, the inhibitor of TRPA1 comprises ruthenium red. In some embodiments, the inhibitor of TRPA1 comprises AM-0902. AM-0902 is chemically described as 1-({3-(2-(4-chlorophenyl)ethyl]-1,2,4-oxadiazol-5-yl}methyl)-7-methylpurin-6-one. Its molecular formula is C₁₇H₁₅ClN₆O₂ and its structural formula is as follows:

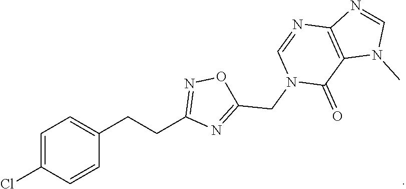 [SMILES: Cn1cnc2ncn(Cc3nc(CCc4ccc(Cl)cc4)no3)c(=O)c21]

In some embodiments, the at least one modulator of oxidative stress comprises a nuclear factor erythroid 2-related factor 2 (NRF2) transcription network activator. NRF2 (or Nfe2l2) is a ubiquitously-expressed transcription factor that governs the expression of a network of antioxidant genes, including Nqo1, Gsta2, and Hmox1. NRF2 is constitutively expressed and translated, but under normal conditions is continually tagged for proteasomal degradation by the E3-ubiquitin ligase KEAP1. “NRF2 transcription network activators” include compounds which activate, stimulate, or induce the NRF2 transcription network.

The at least one modulator of oxidative stress may be selected from the group consisting of: sulforaphane, exemestane, JQ-1, BRD-K32656671, 16,16-dimethylprostaglandin-e2, 16β-bromoandrosterone, gedunin, CA-074-Me, BRD-K67258146, xanthohumol, penicillic acid, BRD-K06817181, GR-235, 15-delta prostaglandin-j2, guggulsterone, pifithrin-mu, parthenolide, BRD-A05680309, MLN-4929, MDM2-inhibitor, benperidol, chaetocin, benzo(a)pyrene, primaquine, SSR-69071, elesclomol, indirubin, JLK-6, flavokavain-b, sappanone-a, 4-hydroxy-2-nonenal, pyrrolidine-dithiocarbamate, and combinations thereof.

In some embodiments, the at least one modulator of oxidative stress comprises sulforaphane, or derivatives or analogs thereof. Sulforaphane is chemically described as 1-isothiocyanato-4-(methanesulfinyl)butane. Its molecular formula is C₆H₁₁NOS₂ and its structural formula is as follows:

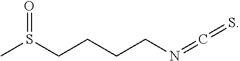

Sulforaphane is found in cruciferous vegetables such as cabbage, broccoli, broccoli sprouts, brussels sprouts, cauliflower, cauliflower sprouts, bok choy, kale, collards, arugula, kohlrabi, mustard, turnip, red radish, and watercress. In the plant, it is present in bound form as glucoraphanin, a glucosinolate. Sulforaphane is often formed from glucoraphanin on plant cell damage via an enzymatic reaction. Sulforaphane may be isolated and purified from natural sources or chemically synthesized by methods known in the art.

Analogs of sulforaphane include, but are not limited to, 6-isothiocyanato-2-hexanone, exo-2-acetyl-6-isothiocyanatonorbomane, exo-2-isothiocyanato-6-methylsulfonylnorbomane, 6-isothiocyanato-2-hexanol, 1-isothiocyanato-4-dimethylphosphonylbutane, exo-2-(1′-hydroxyethyl)-5-isothiocyanatonorborane, exo-2-acetyl-5-isothiocyanoatonorbormane, 1-isothiocyanato-5-methylsulfonylpentane, and cis- or trans-3-(methylsulfonyl)cyclohexylmethylisothiocyanate.

The sulforaphane may be a single enantiomeric species or a racemic mixture. Any ratio of enantiomers of sulforaphane may be present.

In some embodiments, the at least one modulator of oxidative stress comprises exemestane, or derivatives or analogs thereof. Exemestane, which is sold as Aromasin®, is chemically described as 6-methylenandrosta-1,4-diene-3,17-dione. Its molecular formula is C₂₀H₂₄O₂ and its structural formula is as follows:

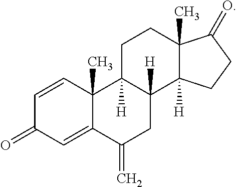

Various methods and intermediates for preparing exemestane are known in the art.

Exemestane is an irreversible, steroidal aromatase inactivator, which is structurally related to the natural substrate androstenedione, that acts as a false substrate for the aromatase enzyme and is processed to an intermediate that binds irreversibly to the active site of the enzyme causing its inactivation. Exemestane lowers circulating estrogen concentrations in postmenopausal women thereby providing a treatment for some postmenopausal patients with hormone-dependent breast cancer.

In some embodiments, the at least one modulator of oxidative stress comprises JQ-1, or a derivative or analog thereof. JQ-1 is a thieno-triazolo-1,4-diazepine, chemically described as (S)-tert-butyl 2-(4-(4-chlorophenyl)-2,3,9-trimethyl-6H-thieno[3,2-f][1,2,4]triazolo[4,3-a][1,4]diazepin-6-yl)acetate. Its molecular formula is C₂₃H₂₅ClN₄O₂S and its structural formula is as follows:

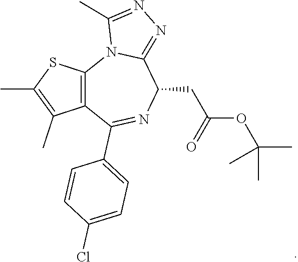 [SMILES: Cc1sc2c(c1C)C(c1ccc(Cl)cc1)=N[C@@H](CC(=O)OC(C)(C)C)c1nnc(C)n1-2]

Derivatives of JQ-1 are known in the art, see for example U.S. Patent Publication No. 2020/0368248, incorporated herein by reference. JQ-1 may be a single enantiomeric species or a racemic mixture. Any ratio of enantiomers of JQ-1 may be present. In some embodiments. JQ-1 is (+)-JQ1.

JQ-1 is a potent bromodomain inhibitor, also referred to as BET bromodomain inhibitors, and acts in displacing bromodomain-containing proteins from acetylated lysine residues on histones. Bromodomain inhibitors have been used to treat cancers, cardiovascular disease, and male fertility.

### 3. COMPOSITIONS

Disclosed herein are compositions comprising a modulator of oxidative stress as described above. The composition may be suitable for administration to a subject, which may be human or non-human.

The modulators of oxidative stress may be incorporated into pharmaceutically acceptable compositions. The pharmaceutical compositions may include a “therapeutically effective amount” or a “prophylactically effective amount” of at least one modulator of oxidative stress. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, or before symptom onset. The prophylactically effective amount will normally be less than the therapeutically effective amount.

The composition may include pharmaceutically acceptable carriers. The term “pharmaceutically acceptable carrier,” as used herein, means a non-toxic, inert solid, semi-solid or liquid filler, diluent, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, flavoring and perfuming agents, preservatives and antioxidants.

The route by which the disclosed compounds are administered and the form of the composition will dictate the type of carrier to be used. The compositions may be in a variety of forms, suitable, for example, for systemic administration (e.g., oral, nasal, sublingual, buccal, implants, or parenteral).

Carriers for systemic administration typically include at least one of diluents, lubricants, binders, disintegrants, colorants, flavors, antioxidants, preservatives, glidants, solvents, suspending agents, wetting agents, surfactants, combinations thereof, and others. All carriers are optional in the compositions. Parenteral administration refers to administration in a manner other than through the digestive tract, such as by intravenous, subcutaneous, intradermal, or intramuscular injection or inhalation.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, dimethyl sulfoxide. N-Methyl-2-Pyrrolidone, dimethylacetamide and phosphate (or other suitable buffer). The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Suitable propellants include propane, butane, isobutane, dimethyl ether, carbon dioxide, nitrous oxide, and combinations thereof. The amount of propellant(s) in a topical composition is typically about 0% to about 95%.

Although the amounts of components in the systemic compositions may vary depending on the type of systemic composition prepared, in general, systemic compositions include 0.01% to 50% of an active compound (e.g., at least modulator of oxidative stress) and 50% to 99.99% of one or more carriers.

Compositions for oral administration can have various dosage forms. For example, solid forms include tablets, capsules, granules, and bulk powders. Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The amount of the carrier employed in conjunction with a disclosed modulator of oxidative stress is sufficient to provide a practical quantity of composition for administration per unit dose of the modulator of oxidative stress. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

### 4. ADMINISTRATION AND DOSAGE

The modulators of oxidative stress of the present disclosure, or compositions thereof, may be administered to a subject by a variety of methods. In any of the uses or methods described herein, administration may be by various routes known to those skilled in the art, including without limitation oral, inhalation, intravenous, intramuscular, subcutaneous, systemic, and/or intraperitoneal administration to a subject in need thereof. In some embodiments, the modulator of oxidative stress or compositions thereof as disclosed herein may be administered by parenteral administration (including, but not limited to, subcutaneous, intramuscular, intravenous, intraperitoneal, intracardiac and intraarticular injections).

In some embodiments, the modulator of oxidative stress or compositions thereof as disclosed herein are administered using perineural injection, systemic injection, or intranasal injection.

The trigeminal nerve innervates tissues of a mammal's (e.g., human) head including skin of the face and scalp, oral tissues, and tissues surrounding the eye. The trigeminal nerve has three major branches, ophthalmic (V₁, sensory), maxillary (V₂, sensory), and mandibular (V₃, motor and sensory) branches. In certain embodiments, the administration comprises administering the modulator of oxidative stress or compositions thereof to or in close proximity to one or more of the trigeminal nerve branches, e.g., by percutaneous, stereotactic administration.

In some embodiments, the administration comprises administering to nasal tissues innervated by the trigeminal nerve, for example, the sinuses, the inferior two-thirds of the nasal cavity, or the nasal septum.

The modulators of oxidative stress and the compositions disclosed herein can be administered therapeutically. In therapeutic applications, the modulators of oxidative stress or a composition thereof is administered to a subject in need thereof in an amount sufficient to elicit a therapeutic effect. An amount adequate to accomplish this is defined as “therapeutically effective dose.” Amounts effective for this use will depend on, e.g., the particular composition of the conjugate regimen administered, the manner of administration, the stage and severity of the disease, the general state of health of the patient, and the judgment of the prescribing physician. For purposes of all of the inventive methods, the dose should be sufficient to affect a therapeutic response in the subject over a reasonable time frame. The modulators of oxidative stress and the compositions disclosed herein can be administered prophylactically. In prophylactic applications, the modulators of oxidative stress or a composition thereof is administered to a subject in need thereof in an amount sufficient to partially or completely delaying onset of one or more symptoms, features, or clinical manifestations of a particular disease or disorder. An amount adequate to accomplish this is defined as “prophylactically effective dose.” Usually, since a prophylactically effective dose is used in subjects prior to or at an earlier stage of disease, or before symptom onset. The prophylactically effective dose will normally be less than the therapeutically effective dose.

Dosage amount and interval may be adjusted individually to provide plasma or local levels of the modulator of oxidative stress which are sufficient to maintain the modulating effects, or minimal effective concentration (MEC). The MEC will vary for each peptide but can be estimated from in vivo and/or in vitro data. Dosages necessary to achieve the MEC will depend on individual characteristics and route of administration. However, bioassays can be used to determine concentrations. In cases of local administration or selective uptake, the effective local concentration of the peptide may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate, precluding toxicity. The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be also used in veterinary medicine for non-human subjects.

Modulators of oxidative stress and compositions disclosed herein can be evaluated for efficacy and toxicity using known methods. For example, the toxicology of a particular composition comprising a modulator of oxidative stress may be established by determining in vitro toxicity towards a cell line, such as a mammalian, and preferably human, cell line. The results of such studies are often predictive of toxicity in animals, such as mammals, or more specifically, humans. Alternatively, the toxicity in an animal model, such as mice, rats, rabbits, dogs, or monkeys, may be determined using known methods. The efficacy of a particular modulator of oxidative stress or composition thereof may be established using several recognized methods, such as in vitro methods, animal models, or human clinical trials, as disclosed herein. When selecting a model to determine efficacy, the skilled artisan can be guided by the state of the art to choose an appropriate model, dose, route of administration and/or regime.

In some embodiments, the effective amount of the modulator of oxidation stress is 0.1-100 mg/kg. The effective amount may be greater than 0.1 mg/kg, 0.5 mg/kg, 1.0 mg/kg, 2.0 mg/kg, 3.0 mg/kg. 4.0 mg/kg. 5.0 mg/kg. 6.0 mg/kg, 7.0 mg/kg, 8.0 mg/kg, 9.0 mg/kg, 10 mg/kg, 15 mg/kg, 20 mg/kg, 25 mg/kg, 30 mg/kg, 35 mg/kg, 45 mg/kg, 50 mg/kg, 60 mg/kg, 70 mg/kg, 80 mg/kg, 90 mg/kg, or 95 mg/kg. The effective amount may be less than 100 mg/kg, 90 mg/kg, 80 mg/kg, 70 mg/kg, 60 mg/kg, 50 mg/kg, 45 mg/kg, 40 mg/kg. 35 mg/kg, 30 mg/kg, 25 mg/kg, 20 mg/kg, 15 mg/kg, 10.0 mg/kg, 9.0 mg/kg, 8.0 mg/kg, 7.0 mg/kg, 6.0 mg/kg, 5.0 mg/kg, 4.0 mg/kg, 3.0 mg/kg, 2.0 mg/kg, 1.0 mg/kg, or 0.5 mg/kg.

The modulators of oxidative stress and compositions disclosed herein may be presented in a single dose or divided doses administered at appropriate intervals, for example, as two, three, four, or more sub-doses per day. Further, the sub-dose itself may be further divided, e.g., into a number of discrete loosely spaced administration.

An effective dose of a modulator of oxidative stress or composition thereof, may be administered alone or in combination with an effective amount of at least one additional therapeutic or prophylactic agent. In some embodiments, effective combination therapy is achieved with a single composition or pharmacological formulation that includes both agents, or with two distinct compositions or formulations, administered at the same time, wherein one composition includes a modulator of oxidative stress as described herein, and the other includes the second agent(s). Alternatively, in other embodiments, the therapy precedes or follows the other agent treatment by intervals ranging from minutes to months.

A wide range of second therapies may be used in conjunction with the modulators of oxidative stress of the present disclosure. The second therapy may be a combination of a second active agent or may be a second therapy not connected to administration of another agent. Such second therapies include, but are not limited to, microvascular decompression, radiofrequency, glycerin rhizotomy, stereotactic radiosurgery, or administration of another agent including, but not limited to, an antidepressant, an anticonvulsant or anxiolytic (e.g., carbamazepine, pregabalin, and gabapentin) and an analgesic.

### 5. EXAMPLES

### Materials and Methods

Human Patients and Samples Patients with trigeminal neuralgia. Chiari malformations, idiopathic normal pressure hydrocephalus, or pseudotumor cerebri were recruited under approved protocols. Cerebrospinal fluid (CSF) from patients was collected into specimen tubes, transferred to 15 mL conical tubes, and subsequently centrifuged for 5 min at 2000 g. The supernatant was then collected, aliquoted, and stored at −80° C. until experimentation.

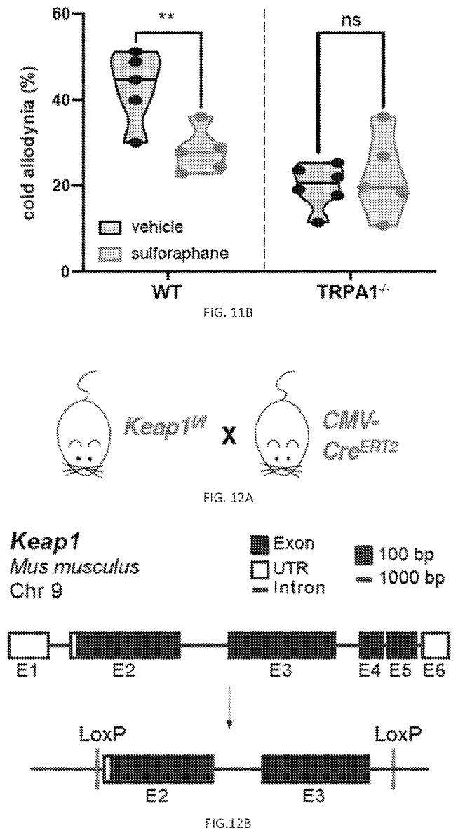

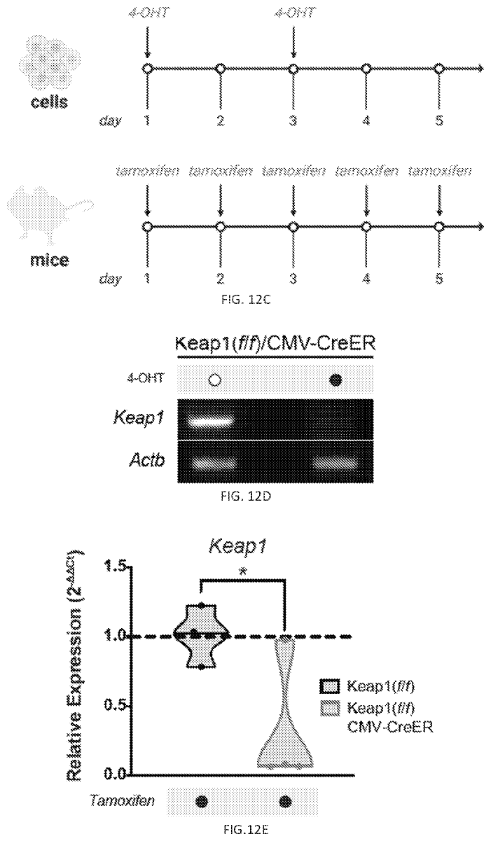

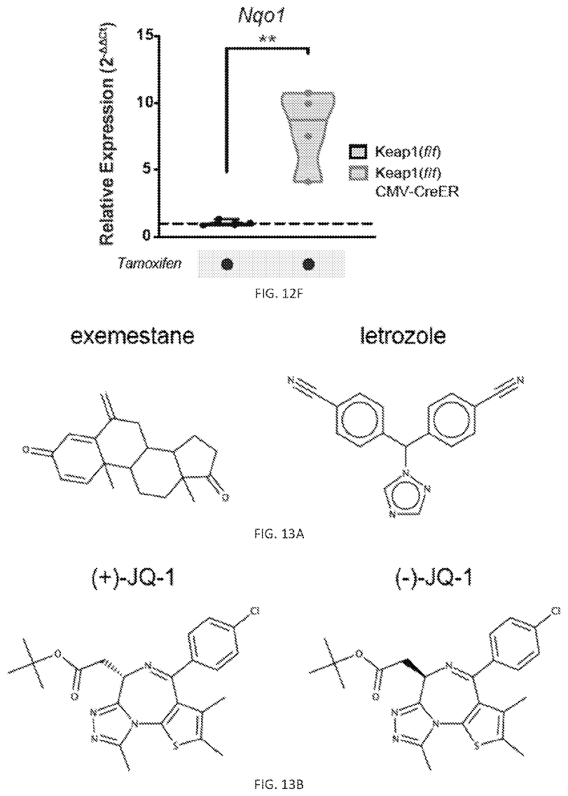

Mice C57BL/6J (Stock #000664) and B6129PF2/J (Stock #100903) WT mice were purchased from Jackson Labs. TRPA1^{-/-} mice were generated as previously described (See Kwan, K. Y. et al. Neuron 50, 277-289 (2006)) and purchased from Jackson Labs. Keap1(f/f) mice were generated as previously described (See Blake, D. J. et al. Am J Resp Cell Mol 42, 524-536 (2010)) Tamoxifen-inducible Keap1(f/f)/CMV-CreER mice were generated by crossing Keap1(f/f) mice with CAG-CreER+ mice as previously described 125. To induce excision of Keap1, Keap1(f/f)/CMV-CreER were injected intraperitoneally with 75 mg/kg tamoxifen once every 24 hrs over 5 consecutive days. Keap1(f/f) (Cre-negative) mice were similarly injected with tamoxifen and served as controls (FIG. 12C). Disruption of Keap1 was confirmed by genomic PCR and by quantitative RNA PCR for Keap1 (FIGS. 12D-E). Nrf2 activation was confirmed by measuring expression of the canonical target NADPH quinone oxidoreductase 1 (Nqo1) by quantitative RNA PCR (FIG. 12F). Behavioral and molecular tests were only performed 7 or more days after the final tamoxifen injection. NRF2^{-/-} mice were generated as previously described (Chan K, et al., Proc National Acad Sci 1996; 93(24):13943-13948, incorporated herein by reference in its entirety) and purchased from Jackson Labs (Stock #017009).

Materials: sulforaphane (Sigma), exemestane (Tocris), letrozole (Cayman), (+)-JQ-1 and (−)-JQ-1 (MedChemExpress), tamoxifen (Sigma), (Z)-4-hydroxytamoxifen (4-OHT) (Sigma), hematoxylin (Sigma), eosin Y (Sigma), osmium tetroxide (Sigma), Fluo-4 AM (Invitrogen), Fura-2 AM (Invitrogen), Fura-2 AM (Invitrogen), ascorbate (Sigma), AM-0902 (Tocris), olive oil (Sigma), and corn oil (Sigma), Dulbecco's Modified Eagle Medium (DMEM) (Gibco), fetal bovine serum (Sigma), penicillin-streptomycin (Gibco), L-glutamine (Gibco), forskolin (Sigma), collagenase/dispase (Sigma), Hanks' Balanced Salt Solution (HBSS) (Gibco), laminin (Roche), poly-D-lysine (Sigma), and protease inhibitor cocktail (Sigma), 10% normal goat serum (Thermo Fisher).

All drugs were freshly prepared in an appropriate solvent immediately prior to beginning each treatment course. Each drug was then aliquoted into individual tubes for each day and stored at −20° C. before thawing at 4° C. just prior to treatment. Sulforaphane was dissolved in 100% DMSO and then diluted to 1% DMSO in saline. Exemestane, letrozole, (+)-JQ-1 and (−)-JQ-1 were dissolved in 100% DMSO and then diluted to 5% DMSO in corn oil. Ascorbate was dissolved directly in saline. AM-0902 was dissolved in 100% DMSO and then diluted to 5% DMSO in olive oil. Tamoxifen was dissolved directly in corn oil. All other compounds were prepared as 50 μl-1,000 μl aliquots and stored at −20° C. before thawing at 4° C. Freeze/thaw cycles were avoided whenever possible.

Constriction of the Maxillary Nerve Constriction of the maxillary division of trigeminal nerve was performed as previously described (See Benedetti, A., et al., Biochimica Et Biophysica Acta Bba—Lipids Lipid Metabolism 620, 281-296 (1980) and Vos, B., et al J Neurosci 14, 2708-2723 (1994)). The procedure was performed under direct visualization and control with a surgical microscope. Mice were first anesthetized with ketamine (100 mg/kg, i.p.) and xylazine (12.5 mg/kg, i.p.) and monitored by pinching the skin between the toes with forceps and monitoring for withdrawal. Mice were restrained with adhesive tape to a sterilized polystyrene board. Upon sufficient anesthesia, the scalp was shaved and an anterior to posterior skin incision was made at the midline to expose the nasal and maxillary bones. The maxillary nerve was exposed and carefully dissected free from the surrounding connective tissue. The distal end of the maxillary nerve was then loosely constricted using 8-0 silk sutures as a ligature. The sutures were tied using a slip knot followed by a normal knot, after which any remaining suture was cut free. The incision was then closed with a 4-0 silk suture. In the sham procedure, the left maxillary nerve was exposed but not constricted. Mice were monitored and rehydrated until fully recovered from anesthesia.

Pharmacologic Treatments Mice were dosed with either sulforaphane (10 mg/kg, i.p.), exemestane (10 mg/kg, i.p.), letrozole (10 mg/kg, i.p.), (+)-JQ-1 (40 mg/kg, i.p.), (−)-JQ-1 (40 mg/kg, i.p.), or ascorbate (100 mg/kg, i.p.) as indicated below. Mice were first dosed daily for two days before surgery and again daily just after behavior testing. For local exemestane treatment, exemestane (5 μL, 25 μg total) was applied directly to the maxillary nerve. Mice received a single dose during surgery. Mice were dosed with AM-0902 (30 mg/kg, p.o.). Mice were treated with AM-0902 were dosed 30 min prior to behavior testing. Mice were dosed with tamoxifen (75 mg/kg, i.p.). Mice were dosed once every 24 hrs over 5 consecutive days. Behavioral and molecular tests were only performed 7 or more days after the final tamoxifen injection.

### Pain Behavior Assays

Mechanical Allodynia Mechanical allodynia was assessed in C57BL/6J, TRPA1^{+/+}, TRPA1^{-/-}, Keap1(f/f), and Keap1(f/f/CMV-CreER mice using a Von Frey filament as previously outlined (See Benedetti, A., et al., Biochimica Et Biophysica Acta Bba—Lipids Lipid Metabolism 620, 281-296 (1980) and Vos, B., et al J Neurosci 14, 2708-2723 (1994)). Animals were placed individually in transparent plastic boxes and allowed to acclimate to the environment for at least 30 min before testing. After habituation, a 0.04 g force Von Frey filament was used to stimulate the territory innervated by the maxillary nerve, including the vibrissal skin pad. Each mouse's response to the filament was scored from 0-4 on the scale below. Each mouse was scored ten times per day. Changes in mechanical allodynia were considered relative to sham- or vehicle-treated animal controls. Behavior was assessed concomitantly or in a blocked manner with consideration for both genotype and treatment.
0—no response
1—non-defensive response to the stimulus (e.g., mouse non-defensively explores filament)
2—withdrawal response (e.g., mouse turns head away from filament)
3—escape/attack response (e.g., mouse moves body away from filament and assumes crouching position against the box wall; actively attacks filament by biting and/or grabbing)
4—asymmetric face grooming (e.g., mouse wipes stimulated facial area in uninterrupted series of at least three face-wash strokes)

Cold Allodynia Cold allodynia was assessed in C57BL/6J, TRPA1^{+/+}, TRPA1^{-/-}, Keap1(f/f), and Keap1(f/f)/CMV-CreER mice using ice-cold acetone as previously outlined (See Yoon, C., et al., Pain 59, 369-376 (1994)). Animals were placed individually in transparent plastic boxes and allowed to acclimatize to the environment for at least 30 min before testing. After habituation, 20 μl of cold acetone was applied to the ligated vibrissal pad skin surface. Cold allodynia was measured as the average time spent wiping the region in a 60 s period, with a maximum of 5 seconds between bouts of wiping. Allodynia was measured three times with 10 min between intervals. Changes in cold allodynia were considered relative to sham- or vehicle-treated animal controls. Behavior was assessed concomitantly or in a blocked manner with consideration for both genotype and treatment.

Western Blotting Western blotting was performed as previously described (See Vasavda, C. et al. Cell Chem Biol 26, 1450-1460.e7 (2019)). Briefly, tissues were homogenized at 4° C. in lysis buffer (pH 7.4 solution of 50 mM Tris-HCl, 150 mM NaCl, 0.1% SDS, 0.5% sodium deoxycholate, and 1% Triton X-100) supplemented with protease inhibitors (Sigma). When dissecting maxillary nerves for immunoblot analysis, nerves were as dissected both as proximally towards the semilunar ganglion and as distal to the skin as possible. Nerves were harvested 11 days after surgery, immediately after the final behavioral test. Lysates were then pulse sonicated and centrifuged at 16,000 g for 10 min at 4° C. Fifteen micrograms of cleared lysate were run on a 4-12% polyacrylamide Bis-Tris gradient gel in running buffer (pH 7.3 solution of 50 mM MES, 50 mM Tris Base, 0.1% SDS, 1 mM EDTA) and then transferred to a PVDF membrane. Membranes were blocked with 5% milk in TBS-T (pH 7.6 solution of 16 mM Tris-HCl, 140 mM NaCl, 0.1% Tween-20) for 1 hr at 25° C. and then incubated with primary antibodies in 3% bovine serum albumin (BSA) (w/v) in TBS-T overnight at 4° C. The following day, membranes were washed with TBS-T, and then incubated with secondary antibodies in 3% BSA (w/v) in TBS-T for 1 hr at 25° C. The following primary antibodies were used: rabbit anti-4-HNE (Abeam ab46545; 1:1.000), rabbit anti-TRPA1 (Novus Biologicals NB110-40763; 1:1,000), rabbit anti-DNP (Millipore Sigma 90451; 1:150), and mouse anti-β-actin (Santa Cruz Biotech sc-47778 HRP; 1:10,000). The following secondary antibodies were used: donkey anti-rabbit IgG (GE Healthcare NA934; 1:10,000) and goat anti-rabbit (Millipore Sigma 90452; 1:300).

Dot Blotting 10 μL of each CSF sample was spotted onto a nitrocellulose membrane in a dot blot manifold and vacuum-dried. Membranes were then blocked with 3% BSA (w/v) in TBS-T for 1 hr at 25° C. and then incubated with a rabbit anti-4-HNE (Abeam ab46545; 1:1,000) primary antibody in 3% bovine serum albumin (BSA) (w/v) overnight at 4° C. The following day, membranes were washed with TBS-T, and then incubated with a donkey anti-rabbit IgG (GE Healthcare NA934; 1:10,000) secondary antibody in 3% BSA for 1 hr at 25° C.

Oxidative Stress Assay % 4-HNE was quantified by Western blot as described above with a rabbit anti-4-HNE (Abeam ab46545; 1:1,000) primary antibody. Protein carbonylation was assessed with the OxyBlot Protein Oxidation Detection Kit as per the manufacturer's instructions. Protein carbonylation was quantified by western blot as described above with a rabbit anti-DNP (Millipore Sigma 90451; 1:150) primary antibody. MDA was quantified with the OxiSelect TBARS Assay Kit as per the manufacturer's instructions. Concentrations below the limit of detection (LOD) were censored and substituted with a constant value of the LOD/42.

Hemoglobin ELISA CSF hemoglobin was quantified with the Human Hemoglobin ELISA Kit as per the manufacturer's instructions. Concentrations below the limit of detection (LOD) were censored and substituted with a constant value of the LOD/√2.

Cell Culture HEK-293 cells were cultured in Dulbecco's Modified Eagle Medium (DMEM), 10% fetal bovine serum, penicillin/streptomycin (100 U/ml), and glutamine (2 mM) in an atmosphere of 5% CO₂ at 37° C. Trigeminal ganglia were pooled into cold DH10 media (90% Dulbecco's Modified Eagle Medium (DMEM). 10% fetal bovine serum, and penicillin/streptomycin (100 U/mL)). Trigeminal ganglia were digested with dispase (5 mg/ml)/collagenase (1 mg/ml) in Hanks' Balanced Salt Solution (HBSS) at 37° C. for 30-45 min. Cells were then triturated, pelleted at 1000 g for 5 min at 25° C., gently rinsed twice in DH10, and then re-suspended in DH10. Dissociated cells were then plated onto glass coverslips coated with poly-D-lysine (0.5 mg/ml) and laminin (10 μg/ml). Neurons were cultured in DH10 supplemented with 50 ng/mL NGF at 37° C. for 12-16 hrs. Immortalized primary human Schwann cells were generated and characterized as previously described (Lehmann H C et al., Stem Cells Dev 2012; 21(3):423-431, incorporated herein by reference in its entirety). Schwann cells were cultured in Dulbecco's Modified Eagle Medium (DMEM), 10% fetal bovine scrum, penicillin/streptomycin (100 U/ml), 2 μM forskolin, and glutamine (2 mM) in an atmosphere of 5% CO₂ at 37° C.

Calcium Imaging and Analysis Calcium imaging and analysis was performed as previously described (See Meixiong, J. et al. Elife 8, (2019)). Briefly, cells were imaged in calcium imaging buffer (CIB; 10 mM HEPES, 1.2 mM NaHCO₃, 130 mM NaCl, 3 mM KCl, 2.5 mM CaCl₂, 0.6 mM MgCl₂, 20 mM glucose, and 20 mM sucrose at pH 7.4 and 290-300 mOsm). To monitor changes in intracellular [Ca²⁺]([Ca²⁺]ᵢ), cells were loaded with either 1 μM Fura 2-AM (HEK293 cells) or 1 μM Fluo 4-AM (trigeminal neurons) for 30 min in the dark at 37° C. in CIB just prior to imaging. With Fura 2-AM, emission at 510 nm was monitored following excitation at both 340 nm and 380 nm. With Fluo 4-AM, emission at 520 nm was monitored following excitation at 488 nm. Cells were identified as positively responding if [Ca²⁺]ᵢ rose by 15% compared to baseline. Damaged, detached, high-baseline, and motion-activated cells were excluded from analysis.

HEK293 cells were plated on poly-D-lysine-coated coverslips and transiently transfected with the vector backbone or constructs encoding WT or mutant TRPA1. Unless otherwise noted, cells were imaged for 30 s to establish a baseline before compounds were added. Vehicle was first applied for 30 s, after which 100 μM iodoacetamide, 1 mM H₂O₂, or 100 μM 4-HNE was applied. CSF from cases and controls was diluted into calcium imaging buffer 1:50 prior to each trial. 50 μM of the non-selective TRP channel inhibitor ruthenium red was applied at the end of every imaging trial.

Neurons were incubated with Fluo-4 AM 12-16 hr after dissociation. Unless otherwise noted, neurons were imaged for 30 s to establish a baseline before compounds were added. Vehicle was first applied for 30 s, after which CSF was applied for 60 s, and then 100 nM capsaicin for 30 s. Randomly pooled CSF from cases or controls was diluted into calcium imaging buffer 1:1 prior to each trial. At the end of every imaging trial, 50 mM KCl was added as a positive control. Percentage activated was determined as described elsewhere herein. In order to distinguish pain- and itch-encoding neurons from other neurons in the culture, the analysis was filtered to neurons that also responded to the TRPV1 agonist capsaicin. Previous studies have demonstrated that TRPA1 and TRPV1 are co-expressed in pain- and itch-encoding sensory neurons and either can serve to unbiasedly identify such neurons.

Schwann cells were plated on poly-D-lysine-coated coverslips. Unless otherwise noted, cells were imaged for 30 s to establish a baseline before compounds were added. Vehicle was first applied for 60 s, after which CSF was applied for 180 s. CSF was diluted into calcium imaging buffer 1:50 prior to each trial.

NRF2/ARE Luciferase Reporter Assay NRF2 activity was monitored with a reporter cell line in which changes in NRF2 activity are coupled to the expression of firefly luciferase (NRF2/ARE Luciferase Reporter HEK293 Stable Cell Line). Luciferase activity was quantified with the Luciferase Assay System as per the manufacturer's instructions. Briefly, cells were plated at 125,000 cells/well in a 24-well plate. The following day, cells were treated with either vehicle or varying doses of sulforaphane, exemestane, or JQ-1 for 6 hours. Cells were then lysed in passive lysis buffer (Promega E1941) supplemented with protease inhibitors (Sigma). Lysates were then cleared at 16,000 g for 15 minutes at 4° C. 15 μL of each lysate was mixed with 5 μL of luciferase assay substrate (Promega). Total light was measured using a luminometer with a 10 second integration time with a delay of 2 seconds. Luciferase measurements were normalized to total protein and reported as normalized to the vehicle condition.

Histology Maxillary nerves from mice were dissected and fixed in cold 4% formaldehyde (v/v) overnight at 4° C. Tissues were then cryoprotected through a series of 10%, 20%, and 30% sucrose (w/v) gradients for 24 hrs each at 4° C. Tissues were then embedded in Optimal Cutting Temperature compound (OCT) and sectioned in 20 μm intervals with a cryostat, after which the sections were dried onto slides and kept at 20° C. Sections were then processed for immunohistochemistry.

Immunohistochemistry was performed as previously described (Vasavda C et al., Cell Chem Biol 2019; 26(10):1450-1460.e7, incorporated herein by reference in its entirety). Briefly, sections were post-fixed with 4% parafomaldehyde for 15 min at 25° C. and then permeabilized with 100% methanol for 7 min at −20° C. The slides were then pre-incubated in blocking solution (10% normal goat serum (ThermoFisher Scientific)) for 30 min at 25° C. Sections were incubated overnight at 4° C. with the appropriate primary antibodies in blocking solution. Sections were washed and incubated with the appropriate secondary antibodies diluted 1:250 in blocking solution for 1 h at 25° C. Tissues were then mounted with ProLong Gold Antifade Mountant with DAPI (Invitrogen).

NRF2/MAP2 immunostaining: Mice were first dosed daily with either sulforaphane (10 mg/kg, i.p.), exemestane (10 mg/kg, i.p.), or the appropriate vehicle for two days before surgical ligation of the maxillary nerve, after which they were dosed every 24 h thereafter. NRF2 immunostaining was performed 24 h after the fourth dose. Antibodies: primary antibodies: rabbit anti-NRF2 (1:100); chicken anti-MAP2 (1:5000); secondary antibodies: goat anti-rabbit (Alexa 568, A-11011 Invitrogen); goat anti-chicken (Alexa 488, A-11039 Invitrogen).

MBP immunostaining: Mice were first dosed daily with either sulforaphane (10 mg/kg, i.p.), exemestane (10 mg/kg, i.p.), JQ-1 (40 mg/kg, i.p.), or the appropriate vehicle for two days before surgical ligation of the maxillary nerve, after which they were dosed every 24 h for 10 days thereafter. MBP immunostaining was performed ten days post-surgery. MBP stains were interpreted by a pathologist to morphologically assess the degree of damage from nerve injury and response to drug treatment. Antibodies: primary antibodies: chicken anti-MBP (1:500); secondary antibodies: goat anti-chicken (Alexa 488, A32931 Invitrogen).

RNA-sequencing RNA sequencing was performed as previously described (See Shin, J. Y. et al. Sci Transl Med 11, eaaw0790 (2019)). To assess the transcriptional signature of JQ-1, total RNA was isolated from three independent human fibroblast lines after 48 hours of treatment with DMSO or 0.25 μM JQ-1 using TRizol and RNeasy isolation columns (Qiagen) as per the manufacturer's instructions. DNA was digested with DNase treatment. All samples had RNA integrity numbers 9.60 or higher as measured with an Agilent 2100 Bioanalyzer. mRNA was enriched by poly-A selection, prepped using an Illumina TruSeq mRNA sample preparation kit, and sequenced by Illumina HiSeq 2000.

Differential Gene Expression and Pathway Enrichment Analyses Differential gene expression analysis was performed using BioJupies 126. BioJupies was used to perform Pathway Enrichment Analyses through Enrichr and to identify the biological processes that are over-represented in the gene set up-regulated by JQ-1 treatment.

DNA/RNA Isolation. PCR, and Quantitative-PCR (q-PCR) Total cellular or tissue DNA/RNA was extracted using the DNeasy Blood & Tissue Kit (Qiagen) for DNA or RNeasy Plus Universal Kit (Qiagen) for RNA per the manufacturer's instructions. PCR was performed with the Platinum Taq DNA Polymerase High Fidelity Kit (Invitrogen), whereas q-PCR was performed with the TaqMan RNA-to-Ct I-Step Kit (Applied Biosystems).

In Silico Candidate Drug Screening The Connectivity Map (CMAP, clue.io) was queried to identify molecules most likely to reproduce the transcriptional signature of both overexpressing Nfe2l2 (the gene encoding NRF2) and genetically silencing Keap1. CMAP scores how well each compound's transcriptional profile matches the query signature from −100 to +100, with a score of +100 indicating a complete match. Molecules with higher scores were interpreted as most likely mimicking upregulating the NRF2 transcriptional network. In the Nfe2l2-queried signature, 187 compounds receive a Connectivity Score above CMAP's recommended cutoff of +90, whereas 114 compounds score above +90 in the Keap1-derived signature. Exemestane and JQ-1 were two overlapping candidate compounds with high scores in both the Nfe2l2-derived and Keap1-derived signatures and were thus prioritized for further validation. Exemestane scored 99.5772 in the Nfe2l2-derived signature and 95.587 in the Keap1-derived signature. JQ-1 scored 99.3935 in the Nfe2l2-derived signature and 99.7503 in the Keap1-derived signature.

Quantification and Statistical Analysis All data were plotted and expressed as the median and range, mean±SEM, or mean±95% CI, as noted. Statistical comparisons were performed using two-tailed unpaired Student's t-tests, Fisher's exact tests, or ANOVA analyses, as noted. Differences were considered significant at P<0.05. All in vivo experiments were performed concomitantly or in a blocked manner with consideration for both genotype and treatment. Mice were excluded from behavioral analysis if they displayed signs of vehicle or drug toxicity or severe, unrelenting pain.

Data and Code Availability JQ-1 RNA-sequencing from fibroblasts treated with JQ-1 are available at Gene Expression Omnibus (GEO) accession #GSE130313 or at ncbi.nlm.nih.gov/geo/query/acc.cgi?acc=GSE130313. JQ-1 transcriptomic analyzes and Compound Connectivity Scores for Nfe2l2-derived and Keap1-derived transcriptome signatures are available at Mendeley with the digital object identifier 10.17632/67p3n9t437.1 or at data.mendeley.com/datasets/67p3n9t437/1.

### Example 1

### Patients and a Mouse Model of Trigeminal Neuralgia Exhibit Increased Oxidative Stress

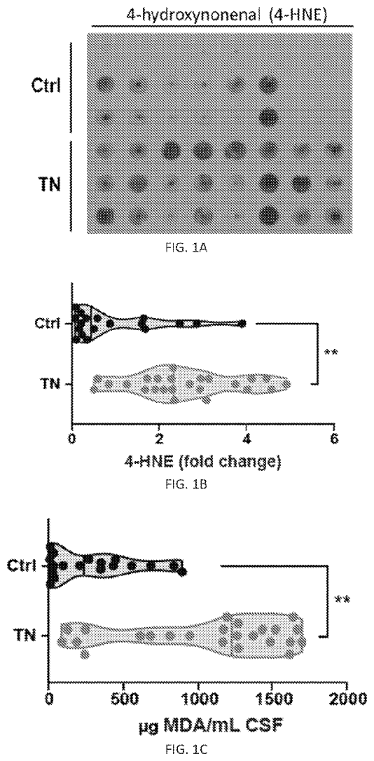

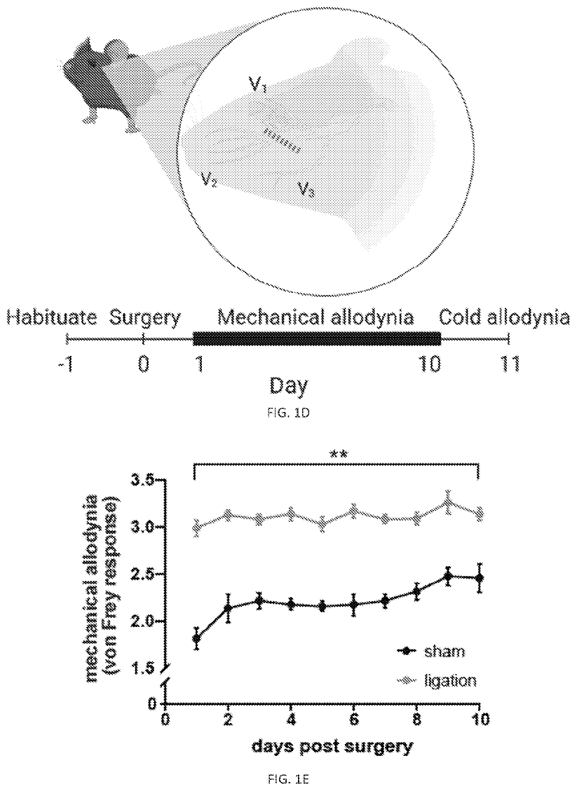

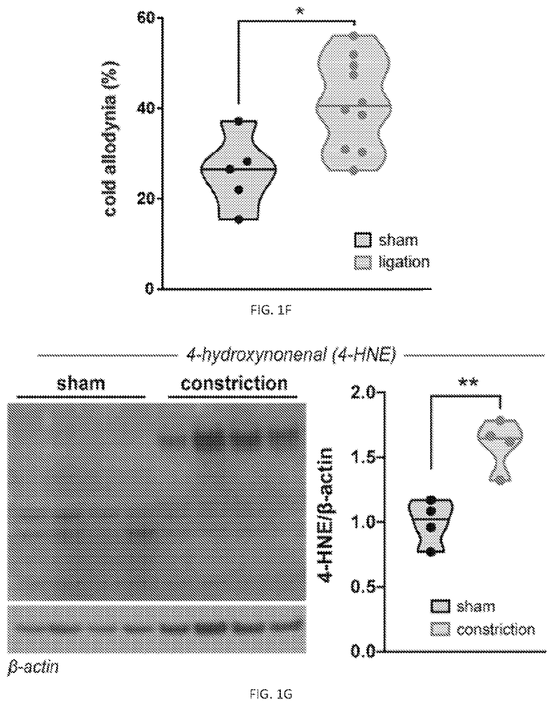

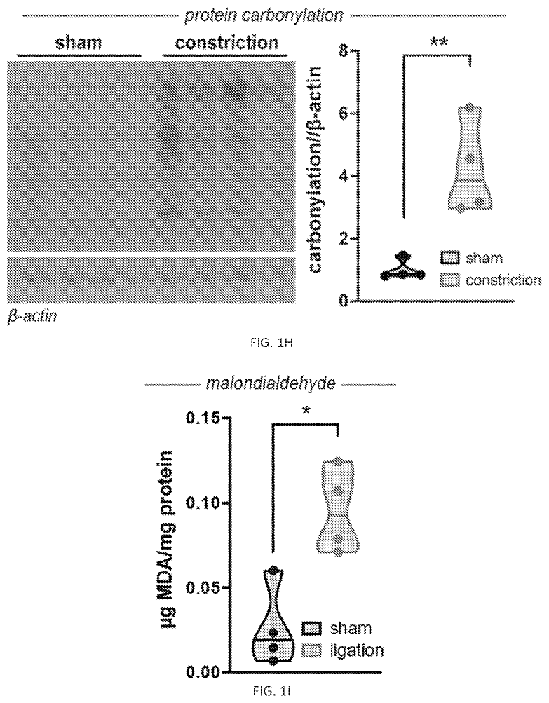

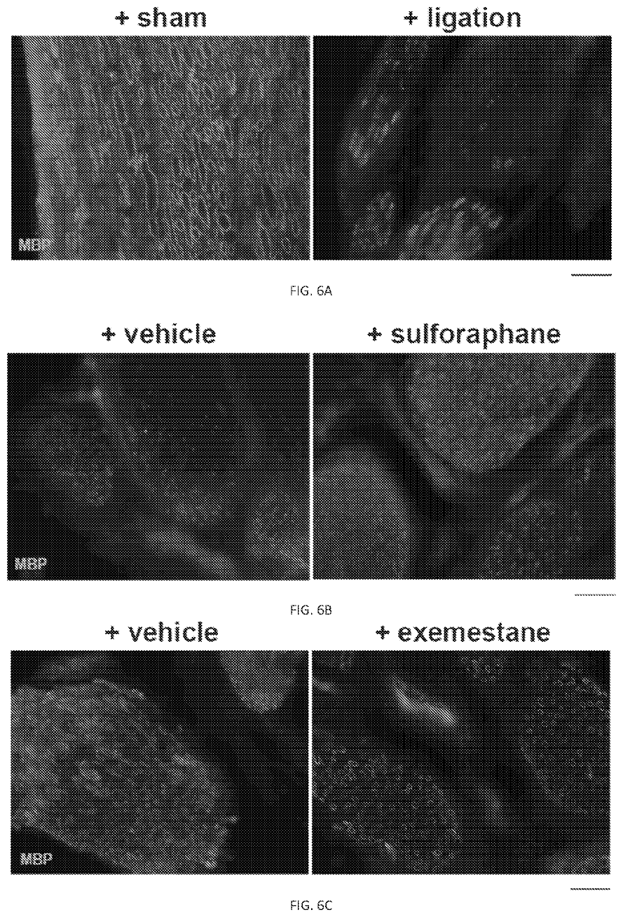

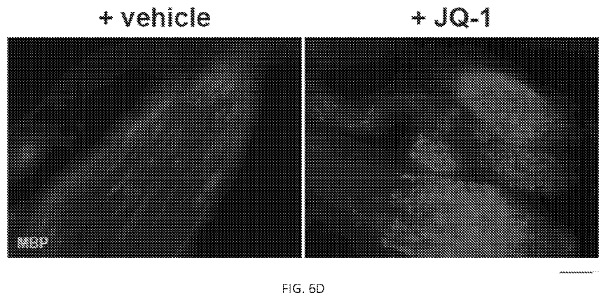

Microvascular decompression requires a craniotomy, presenting a unique opportunity to sample cerebrospinal fluid (CSF) from patients with trigeminal neuralgia. Patients' CSF was evaluated for evidence of oxidative stress by measuring 4-hydroxynonenal (4-HNE) and malondialdehyde, two major products of lipid peroxidation. Compared to CSF collected from patients undergoing posterior fossa craniectomies (to release Chiari malformations), shunts (to relieve normal pressure hydrocephalus or pseudotumor cerebri), or lumbar punctures (to ease pseudotumor cerebri), both 4-HNE and malondialdehyde were markedly elevated in CSF from patients with trigeminal neuralgia (FIG. 1A-IC). CSF 4-HNE and malondialdehyde did not correlate with CSF hemoglobin, suggesting that they were not blood contaminants during microvascular decompression (FIGS. 5A-5B). 4-HNE and malondialdehyde were unlikely to be artifacts of surgery or anesthesia either, since both were still elevated in patients with trigeminal neuralgia compared to controls who underwent similar posterior fossa craniectomies (FIGS. 5C-5D). Instead, the accumulation of CSF 4-HNE and malondialdehyde in trigeminal neuralgia likely reflected elevated oxidative stress.

To monitor and manipulate the influence of oxidative stress in trigeminal neuralgia, a mouse model was used. In this model, branches of the trigeminal nerve are chronically constricted with a loose ligature. Constricting branches of the trigeminal nerve in mice elicited allodynia and hyperalgesia in the region innervated by the damaged nerve (FIG. 1D), as previously reported.

Constricting the maxillary division of trigeminal nerve in mice elicited both mechanical and cold allodynia (FIGS. 1E-1F). Mice exhibited heightened nocifensive behavior to crude touch with a von Frey filament (FIG. 1E) and to application of ice-cold acetone to the affected vibrissal pad skin surface (FIG. 1F). Like the patient cohort, this mouse model also exhibited notable oxidative stress. Ligating the maxillary nerve resulted in marked increases in 4-HNE (FIG. 1G), protein carbonylation (FIG. 1H), and malondialdehyde (FIG. 1I), mirroring the oxidative stress in CSF from trigeminal neuralgia patients. In comparing constricted maxillary nerves to their sham surgical counterparts in mice, sham treated nerves demonstrate normal nerve architecture with axons surrounded by myelin sheaths stained by myelin basic protein (MBP). The ligated nerves exhibit a decreased density of MBP-positive myelin sheaths. Moreover, many of the myelin sheaths showed disrupted architecture with a loss of their circular morphology (FIG. 6A).

### Example 2

### TRPA1 is Activated by Reactive Oxygen Species and Mediates Trigeminal Neuropathic Pain

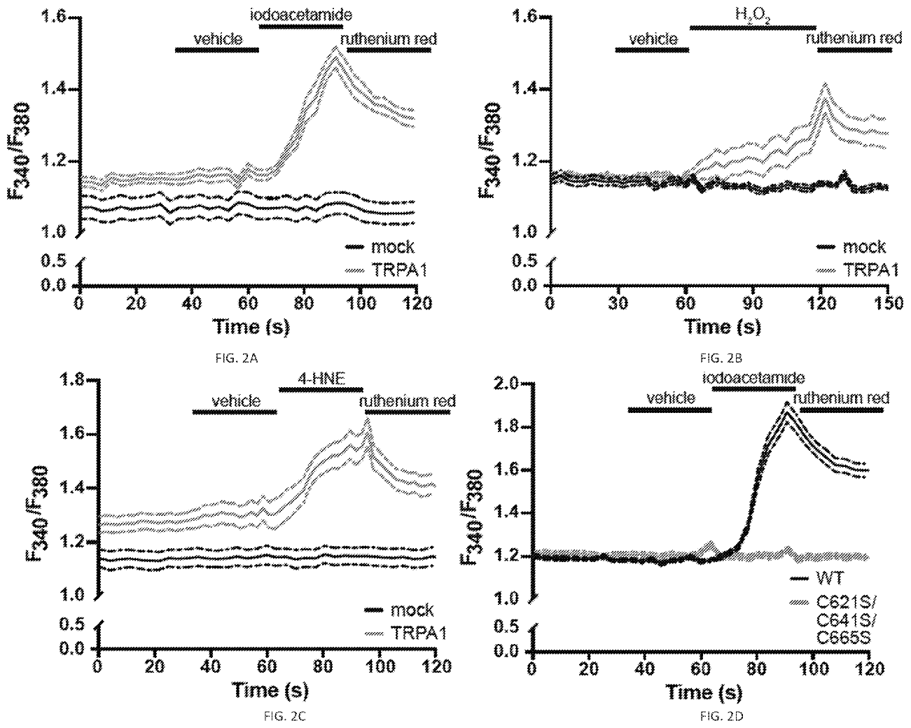

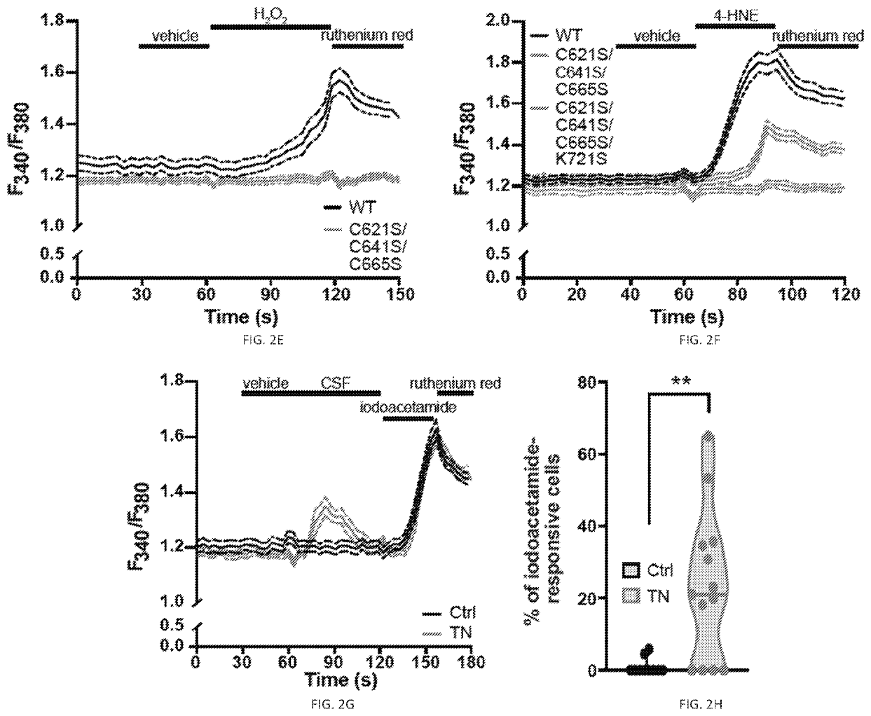

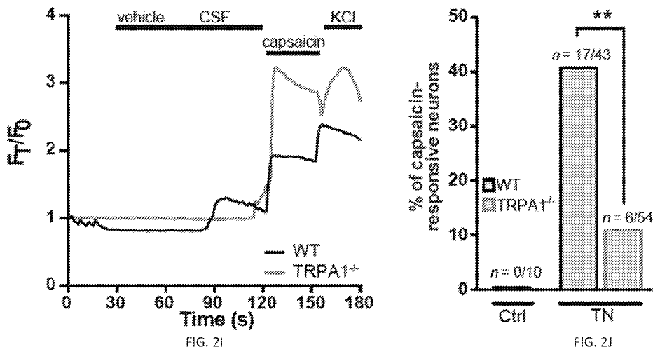

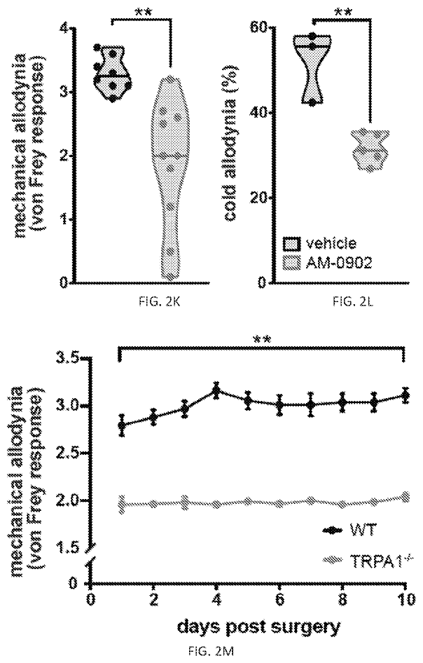

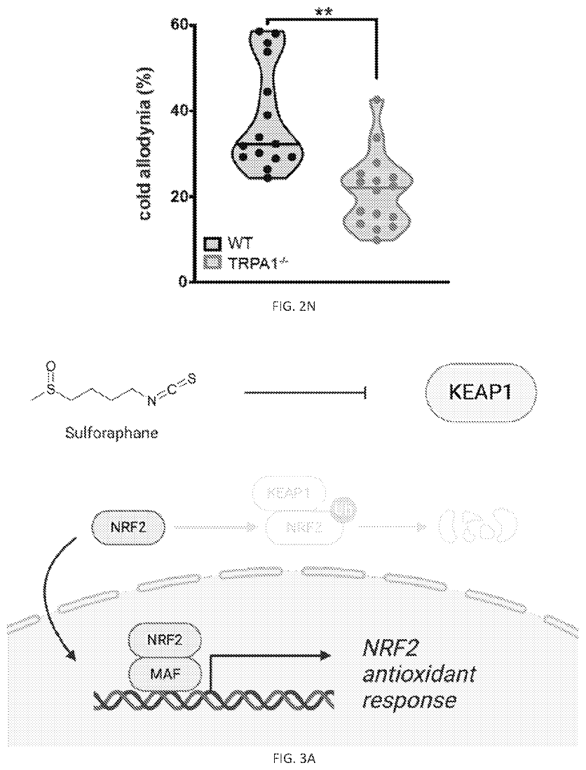

As ROS accumulate in the constrictive mouse model of trigeminal neuralgia, one mechanism by which they may elicit pain is by activating the pain-transducing channel TRPA1. TRPA1 is a non-selective cation channel located at the plasma membrane of both pain- and itch-encoding sensory neurons. TRPA1 is a principal sensor of noxious cold but is also capable of sensing environmental irritants and reactive molecules like iodoacetamide. To evaluate if and how ROS activate TRPA1, TRPA1 was expressed in human embryonic kidney (HEK) 293 cells and changes in intracellular calcium were monitored in response to H₂O₂ and 4-HNE. Both H₂O₂ and 4-HNE activated cells expressing TRPA1 (FIGS. 2A-2C), consistent with earlier evidence that TRPA1 is sensitive to redox-active molecules. The non-selective TRP channel inhibitor ruthenium red was able to quench the calcium response, suggesting that H₂O₂ and 4-HNE initiate calcium signaling through TRPA1 and not downstream effectors. Neither iodoacetamide, H₂O₂, nor 4-HNE elicited a calcium response from cells transfected with the vector backbone alone.

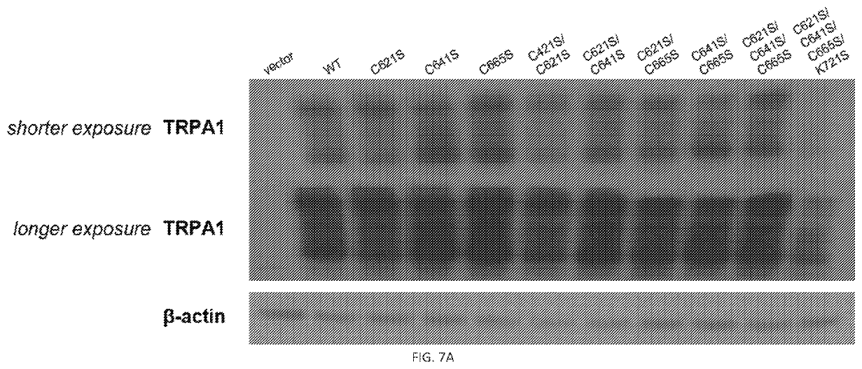

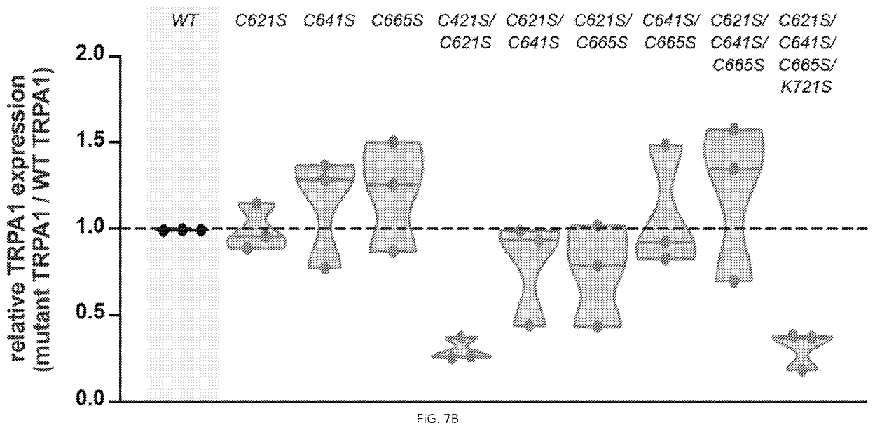

ROS directly activated TRPA1 by covalently bonding or modifying a network of cysteine and lysine residues within the channel. Extensive work by several groups has pinpointed a collection of key residues in TRPA1, including Cys421, Cys621, Cys641, Cys665, and Lys721. To test which residues among these sense H₂O₂ and 4-HNE, each was mutated individually and in various combinations (FIGS. 7A and 7B). Triply mutating cysteines Cys621, Cys641, Cys665 to serine rendered TRPA1 insensitive to both iodoacetamide and H₂O₂ (FIGS. 2D-2E). As previously described, this triple mutant was still activated by 4-HNE but was no longer sensitive after mutating nearby Lys721 (FIG. 2F). These four residues position TRPA1 as both a key sensor and transducer of oxidative stress.

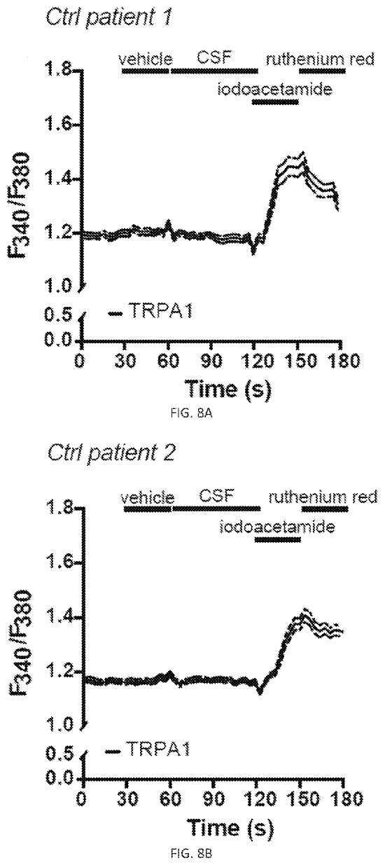

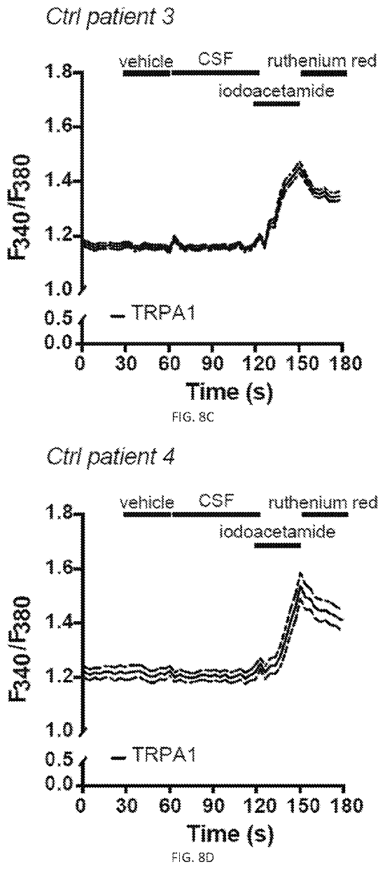

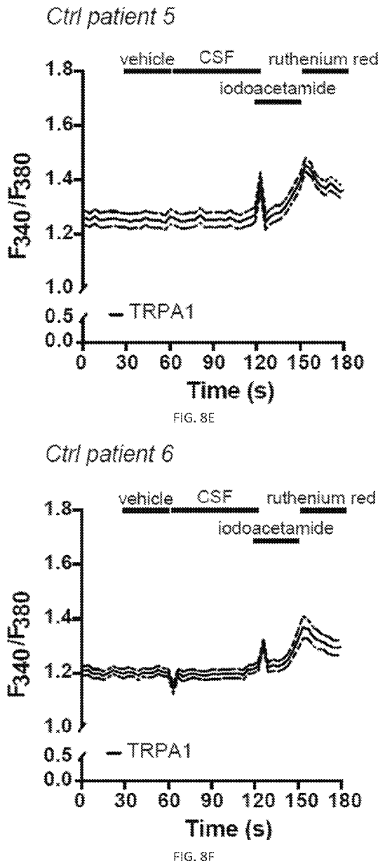

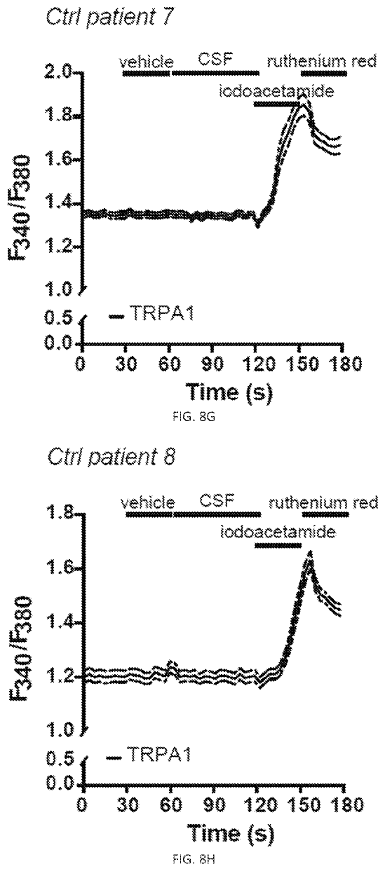

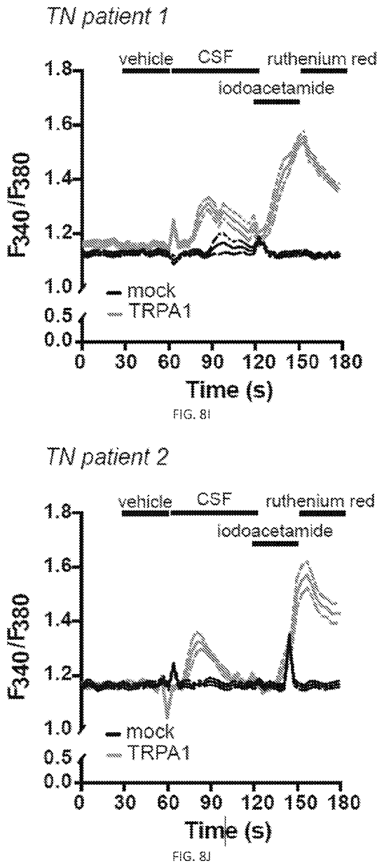

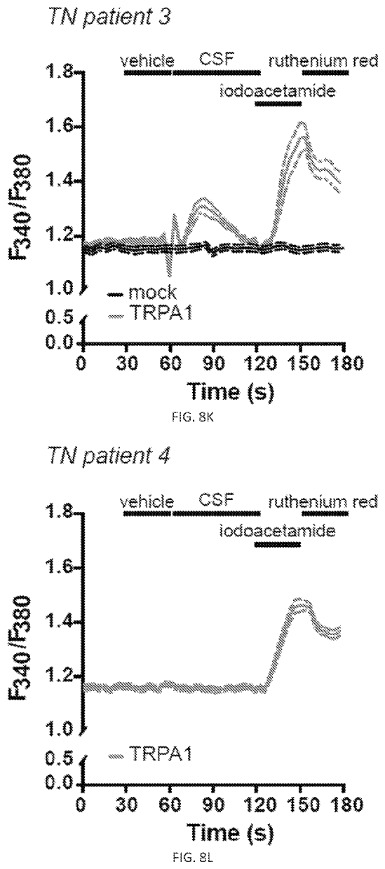

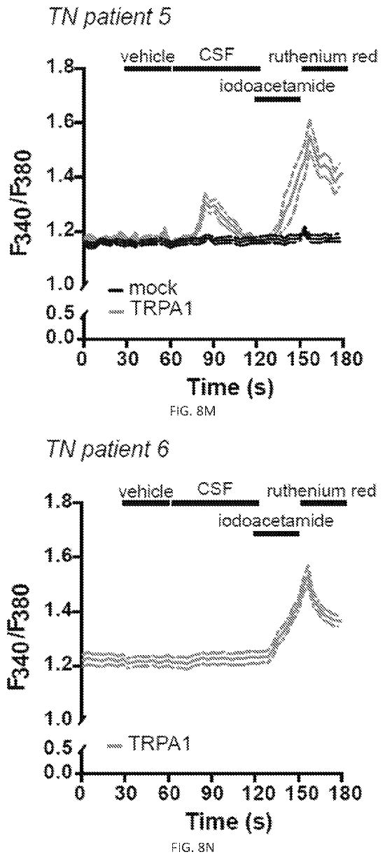

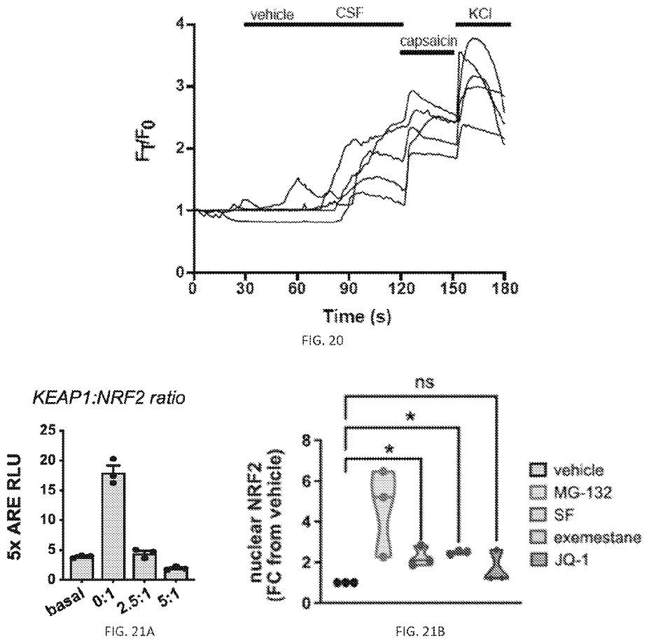

Of the 13 trigeminal neuralgia cases screened, CSF from all but 4 activated TRPA1-expressing HEK cells. In contrast, CSF from 9 out of 11 control patients did not activate TRPA1-expressing cells (P=0.0188; FIGS. 8A-8P). Moreover, CSF from trigeminal neuralgia patients activated cells expressing TRPA1 but not cells transfected with the vector backbone alone (FIGS. 2G and 8I-8P), suggesting that components of the CSF specifically activate TRPA1. CSF from each trigeminal neuralgia patient activated over 20% of iodoacetamide-responsive cells, whereas CSF from control patients activated less than 2% of iodoacetamide-responsive cells (P=0.0031) (FIGS. 2H and 20). To confirm that CSF from trigeminal neuralgia patients can also activate TRPA1 in its native, neuronal environment, CSF from trigeminal neuralgia patients and controls was applied to wild type (WT) and TRPA1-null (TRPA1^{-/-}) trigeminal neuronal cultures. To distinguish pain- and itch-encoding neurons from other neurons in both WT and TRPA1^{-/-} cultures, the analysis was filtered to neurons that also responded to the transient receptor potential cation channel subfamily V member 1 (TRPV1) agonist capsaicin. Previous studies have demonstrated that 97% of TRPA1-positive sensory neurons express TRPV1, and thus TRPV1 can serve to unbiasedly identify pain- and itch-encoding sensory neurons in the absence of TRPA1 expression. By this definition, CSF from trigeminal neuralgia patients activated almost 40% of WT capsaicin-responsive neurons but only 11% of TRPA1^{-/-} neurons (P=0.0016) (FIGS. 2I-2J). CSF from control patients did not activate any sampled WT neurons, further suggesting that unique components of CSF from trigeminal neuralgia patients can activate endogenous TRPA1.

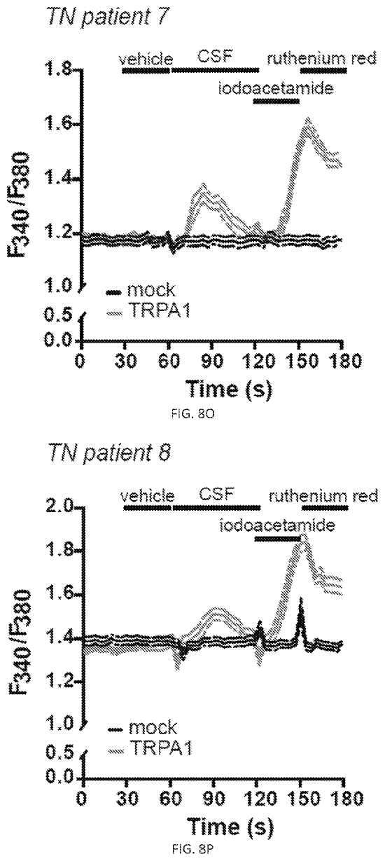

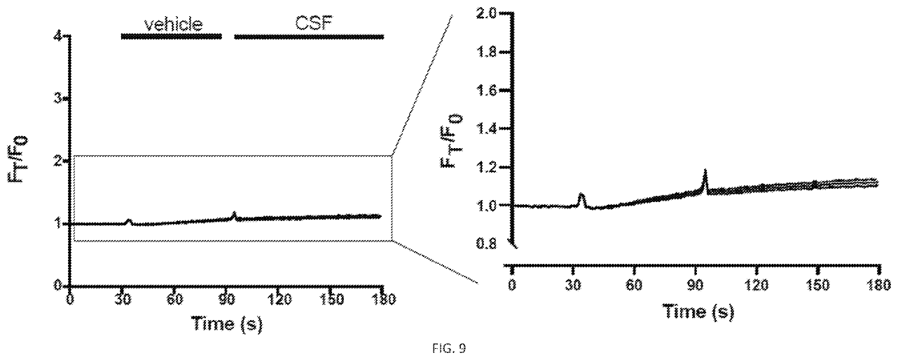

Whether patient CSF directly activates cultured human Schwann cells was tested by monitoring changes in intracellular calcium upon applying CSF. Whereas patient CSF activates TRPA1-positive neurons, no change was observed in calcium signaling in Schwann cells (FIG. 9).

WT mice treated with the TRPA1 inhibitor AM-0902 exhibited reduced mechanical and cold allodynia (FIGS. 2K-2L). Mice that genetically lack TRPA1 exhibited less mechanical and cold allodynia as well (FIGS. 2M-2N). TRPA1^{-/-} males and females both exhibit less mechanical and thermal allodynia (FIGS. 10A-10D).

### Example 3

### Activating NRF2 Attenuates Trigeminal Neuropathic Pain and Oxidative Stress

A principal cellular defense mechanism against oxidative or electrophilic stress is activation of the NRF2-antioxidant response. NRF2 (or Nfe2l2) is a ubiquitously-expressed transcription factor that governs the expression of a network of antioxidant genes, including Ngo1, Gsa2, and Hmox1. As a master regulator of the cellular redox state, NRF2 is tightly regulated. NRF2 is constitutively expressed and translated, but under normal conditions is continually tagged for proteasomal degradation by the E3-ubiquitin ligase KEAP1. Like TRPA1, KEAP1 harbors several redox-sensitive cysteines that are readily modified by electrophiles and oxidants. Oxidation of these cysteines by ROS inhibits KEAP1, stabilizing NRF2 such that it can then translocate to the nucleus to induce expression of antioxidant and cytoprotective genes.

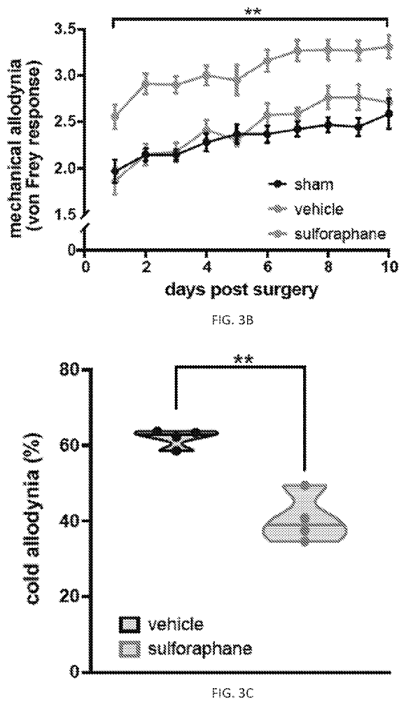

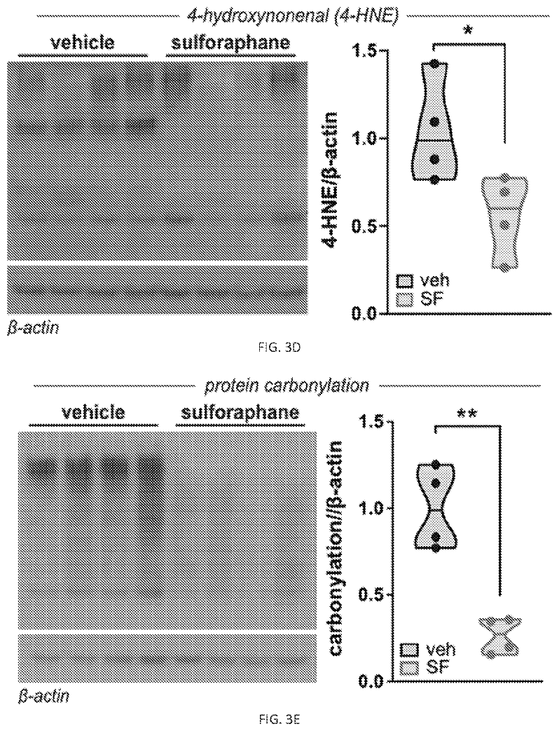

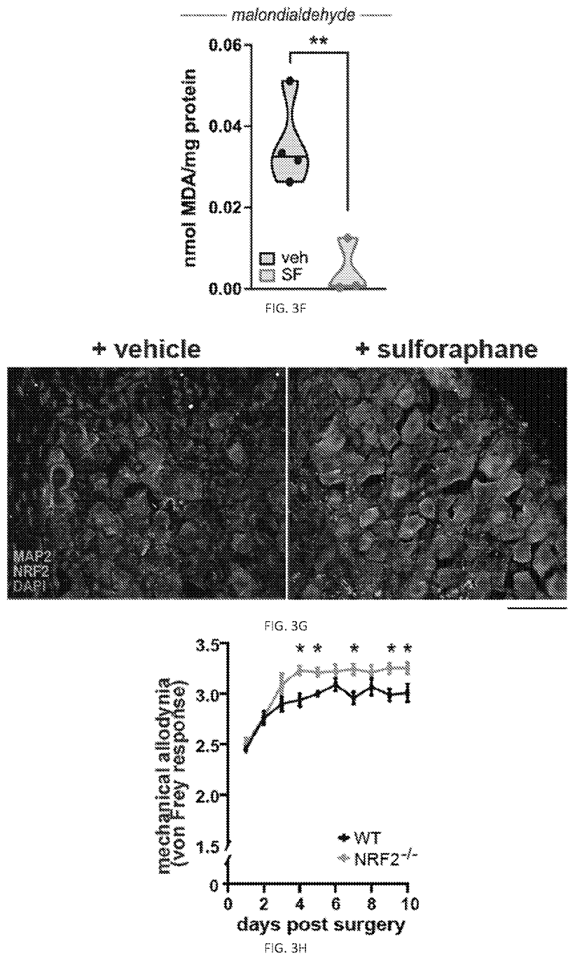

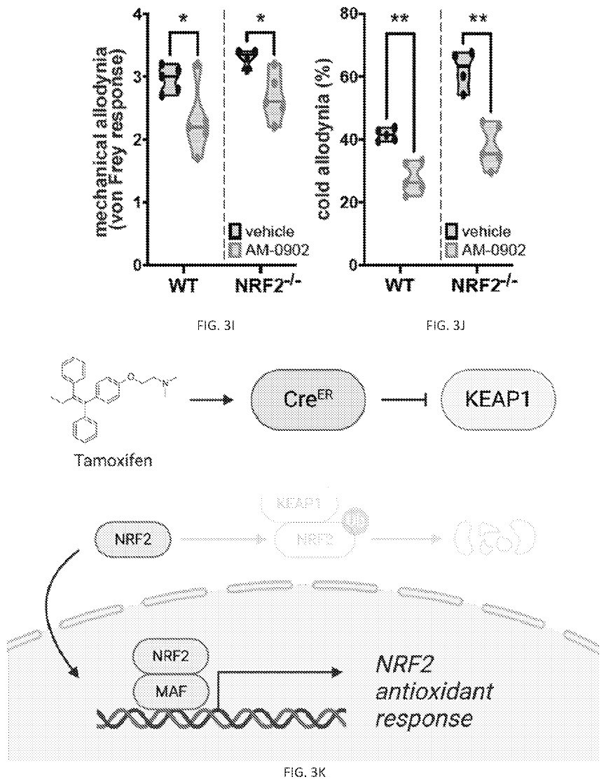

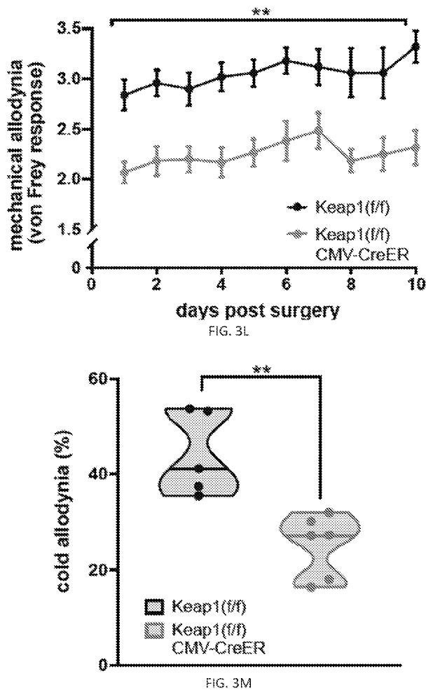

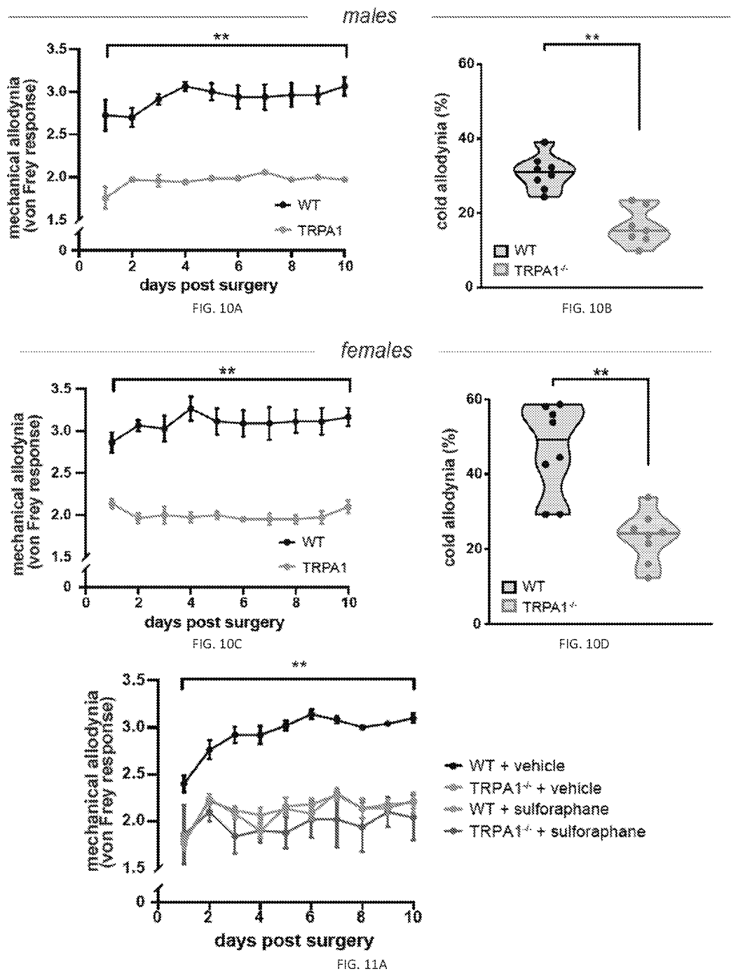

Mice treated with the KEAP1 inhibitor sulforaphane were less sensitive to both crude touch and cold than vehicle-treated mice after constricting the maxillary nerve (FIGS. 3B-3C). However, sulforaphane did not lower mechanical or cold allodynia in TRPA1^{-/-} mice any further compared with genetic deletion of TRPA1 alone, suggesting that oxidative stress contributes to pam upstream of, and possibly through, TRPA1 (FIGS. 11A-11B). Pre-treating mice with sulforaphane limited oxidative stress after nerve ligation and reduced the levels of 4-HNE (FIG. 3D), protein carbonylation (FIG. 3E), and malondialdehyde (FIG. 3F). By immunohistochemistry, it was found that sulforaphane robustly increased NRF2 expression in neurons of the trigeminal ganglion (FIG. 3G). Myelin basic protein stains of vehicle-treated nerves after ligation identified mainly well-myelinated large caliber axons. In comparison, sulforaphane-treated nerves exhibit more normal architecture with myelinated axons of varying sizes (FIG. 6B).

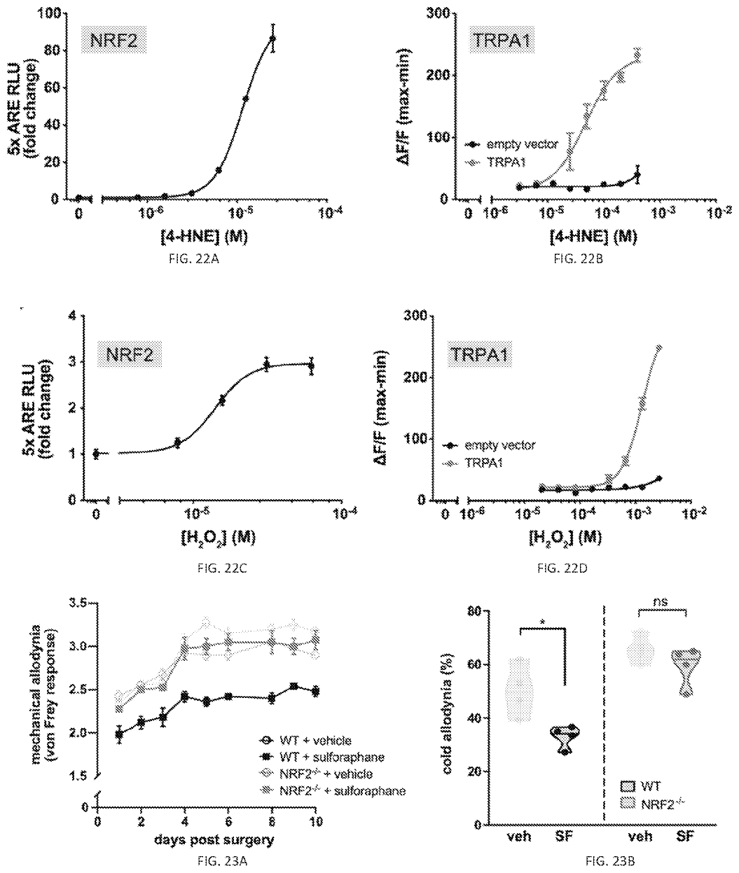

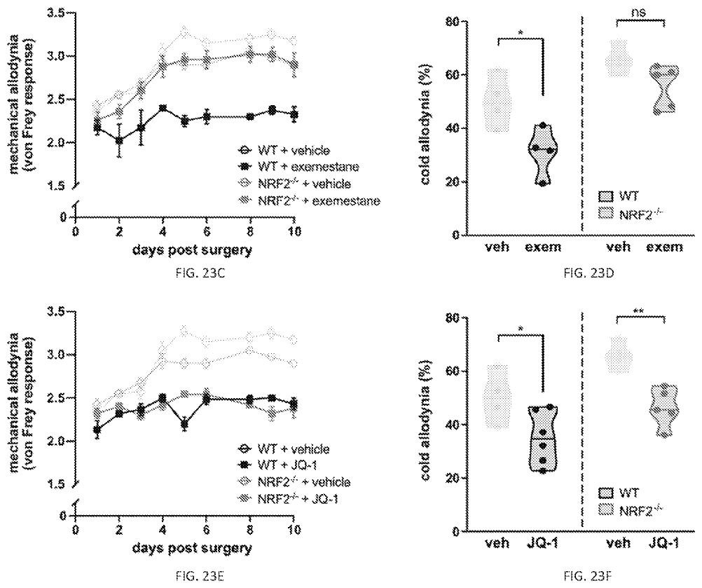

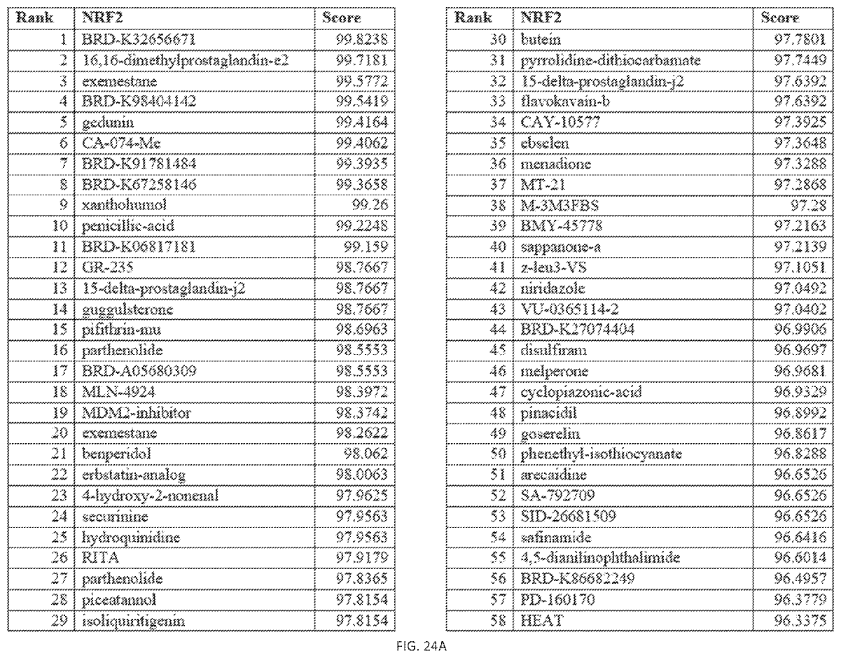

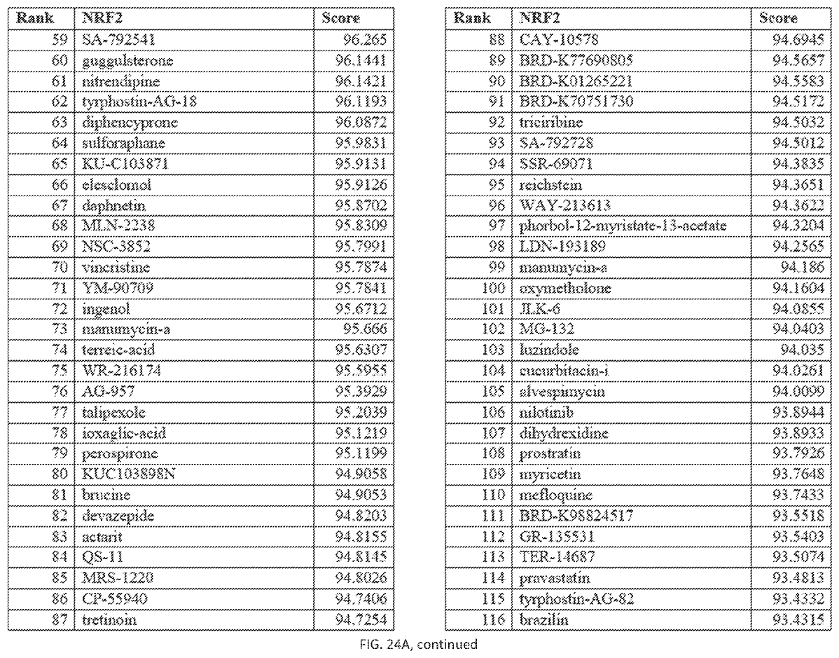

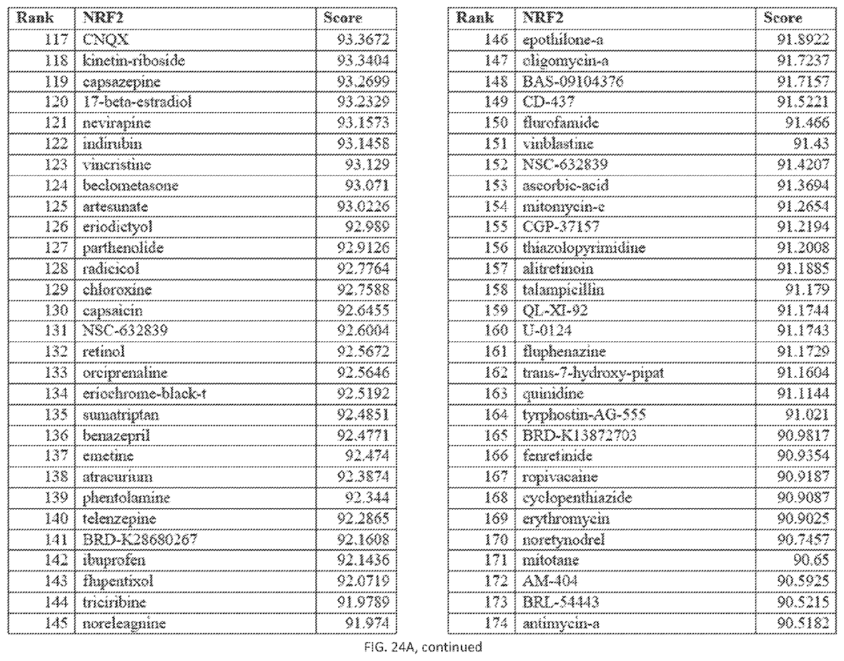

NRF2^{-/-} mice exhibited greater mechanical and cold allodynia after ligation of the maxillary nerve compared to WT mice (FIG. 3H). Just as with WT mice however, treating NRF2^{-/-} mice with the TRPA1 antagonist AM-0902 lowered both mechanical and cold allodynia, suggesting that oxidative stress is epistatic to TRPA1, with TRPA1 transducing the increased oxidative stress into hyperalgesia and allodynia (FIGS. 3I-J). NRF2 is also activated by lower concentrations of 4-HNE and H₂O₂ than TRPA1, suggesting that TRPA1 is activated during periods of greater oxidative stress. Specifically. 4-HNE stimulates NRF2 at an EC₅₀ of 12 μM (95% CI 10-15 μM) and TRPA1 at 48 μM (95% CI 32-117 μM), whereas H₂O₂ triggers NRF2 at an EC₅₀ of 14 μM (95% CI 1-17 μM) and TRPA1 at 133 μM (95% CI 117-167 μM) (FIGS. 22A-22D).

Though sulforaphane activates NRF2, it can exert additional confounding pharmacologic activities. The NRF2 transcriptional network was genetically augmented by eliminating KEAP1 altogether. Keap1-floxed (Keap1(f/f)) mice that contain a tamoxifen-inducible Cre recombinase were generated (FIGS. 3K and 12A-12B). Fibroblasts isolated from these mice were treated with vehicle or 4-hydroxytamoxifen and subsequently genotyped to confirm that Keap1 was retained unless inducibly targeted and excised (FIGS. 12C-12D). When treated with tamoxifen, mice harboring both Cre recombinase and floxed Keap1 alleles exhibited loss of Keap1 with a concomitant increase in Nqo1, a canonical NRF2 target gene (FIGS. 12E-12F). Compared to mice lacking Cre recombinase, mice harboring both Cre and floxed Keap1 alleles exhibited significantly less mechanical and cold allodynia after nerve ligation (FIGS. 3J-3K). Eliminating Keap1 generally matched the response observed with sulforaphane, though the analgesic effect of eliminating Keap1 was slightly more consistent across days than with sulforaphane (FIGS. 3B and 3J).

### Example 4

### Drug Repositioning Identifies NRF2 Network Modulators as Potential Treatments for Trigeminal Neuropathic Pain

To screen for candidate compounds, a transcriptome-guided drug discovery scheme termed transcriptome reversal was used. Transcriptome reversal posits that if a dysregulated transcriptome drives a particular disease, then correcting the transcriptome back toward a normal state may be therapeutic. To reverse the dysregulated transcriptome, the pathologic genetic signature is compared to the transcriptomes of cells treated with different small molecules. Molecules with transcriptome signatures that anticorrelate with the disease signature are prioritized for further validation. The Connectivity Map (CMAP) provides publicly available expression signatures derived from cell lines treated with thousands of small molecules. Transcriptomic approaches that have leveraged CMAP and other resources have successfully identified targeted therapeutics for cancers, as well as diabetes, inflammatory bowel disease, and neurodevelopmental disorders.

To identify therapeutic candidates that induce the NRF2 transcriptional network, compounds that best mimic the transcriptional signature of overexpressing Nfe2l2 and genetically silencing Keap1 were queried (FIGS. 3A and 3K).

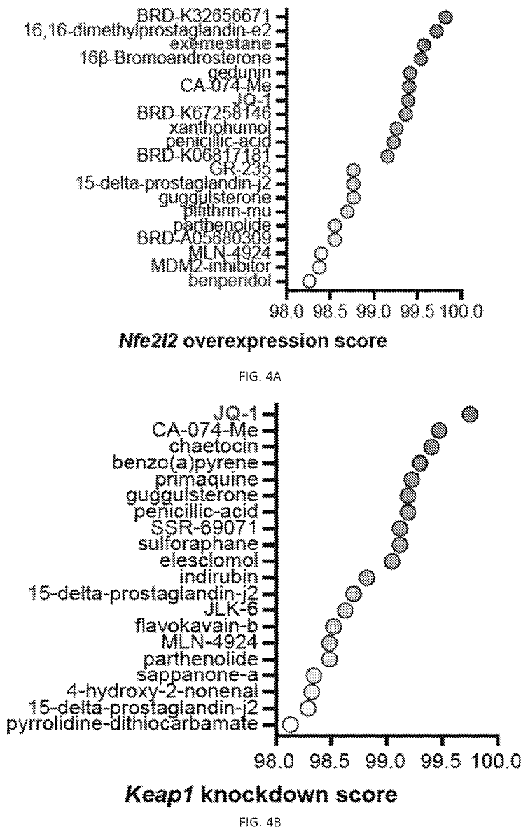

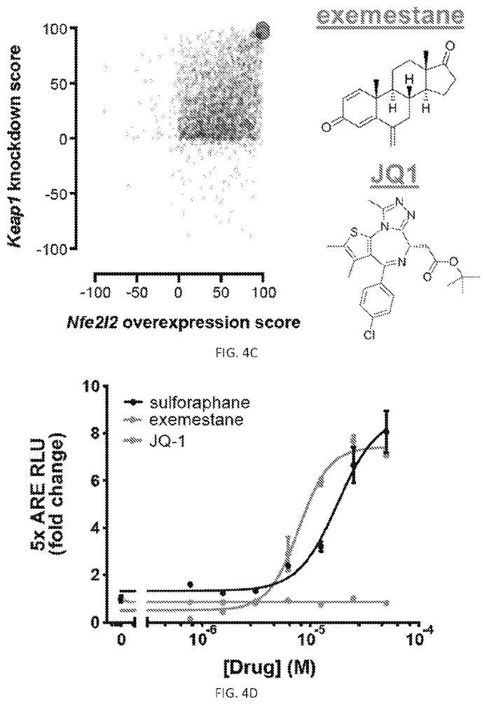

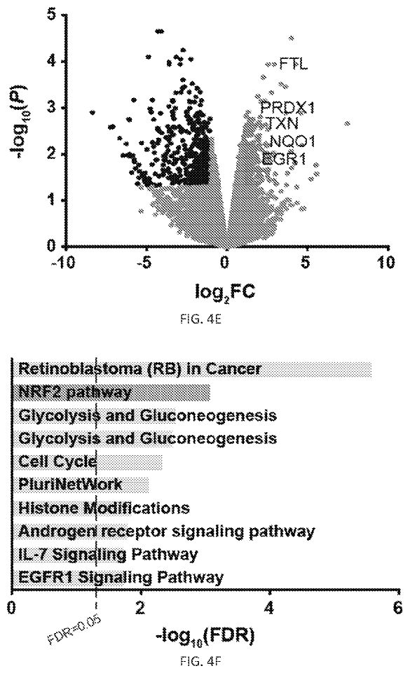

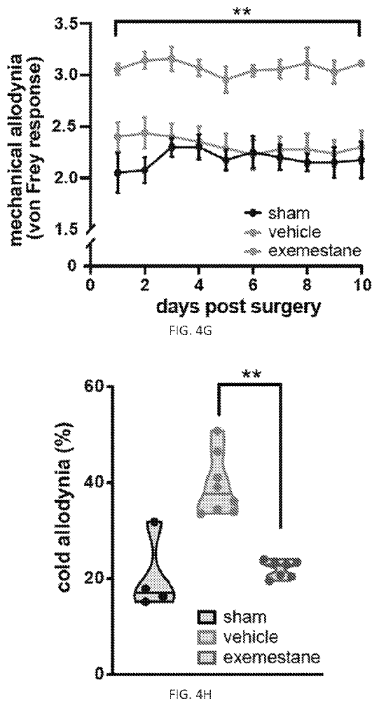

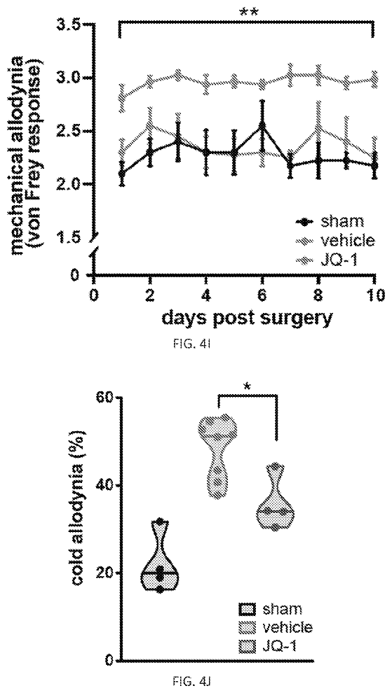

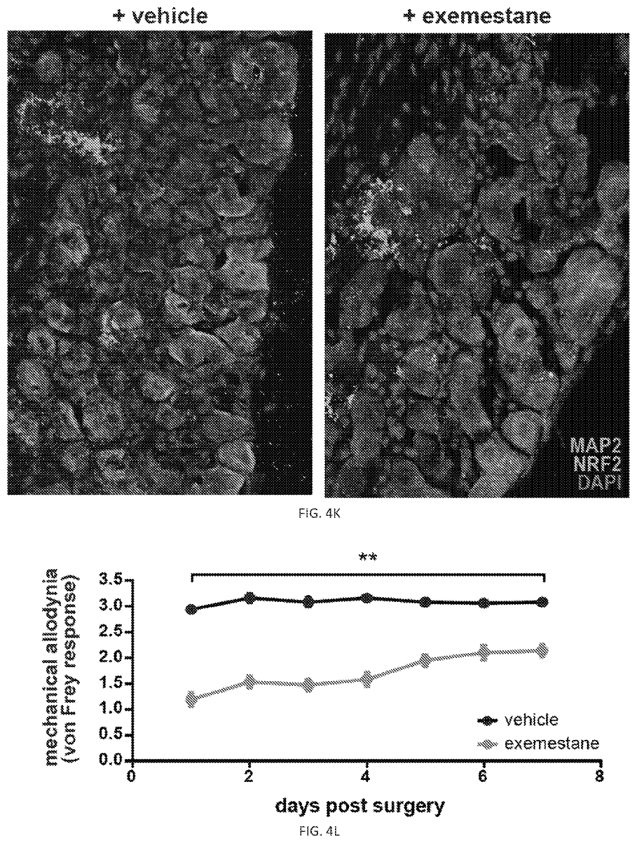

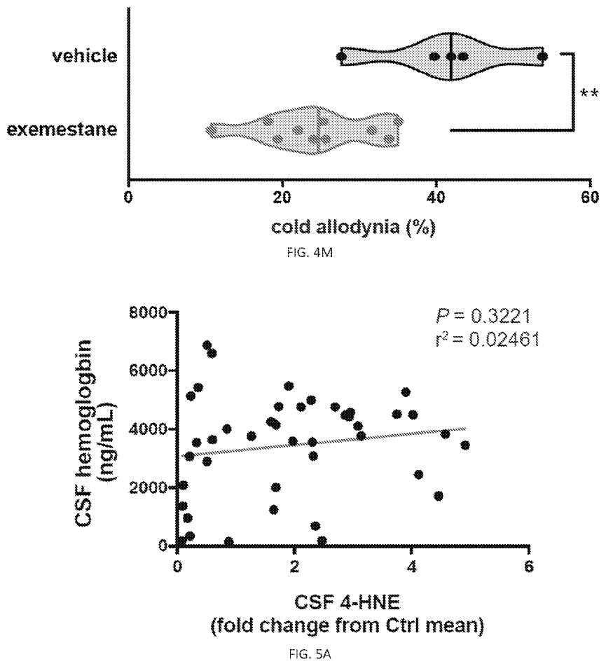

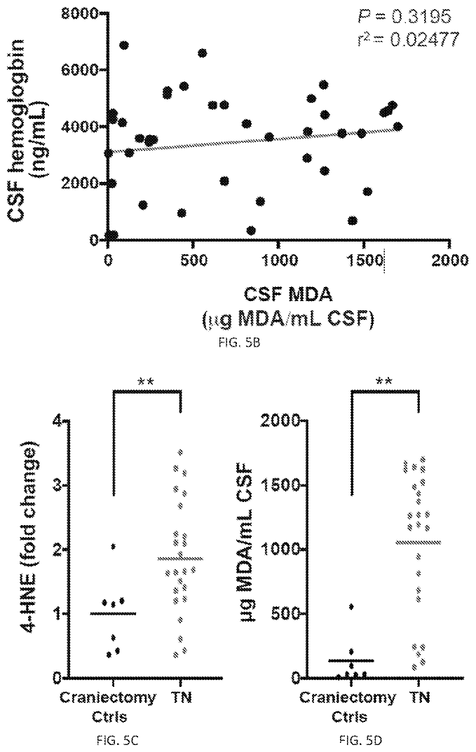

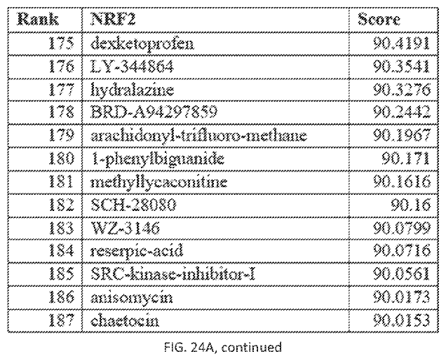

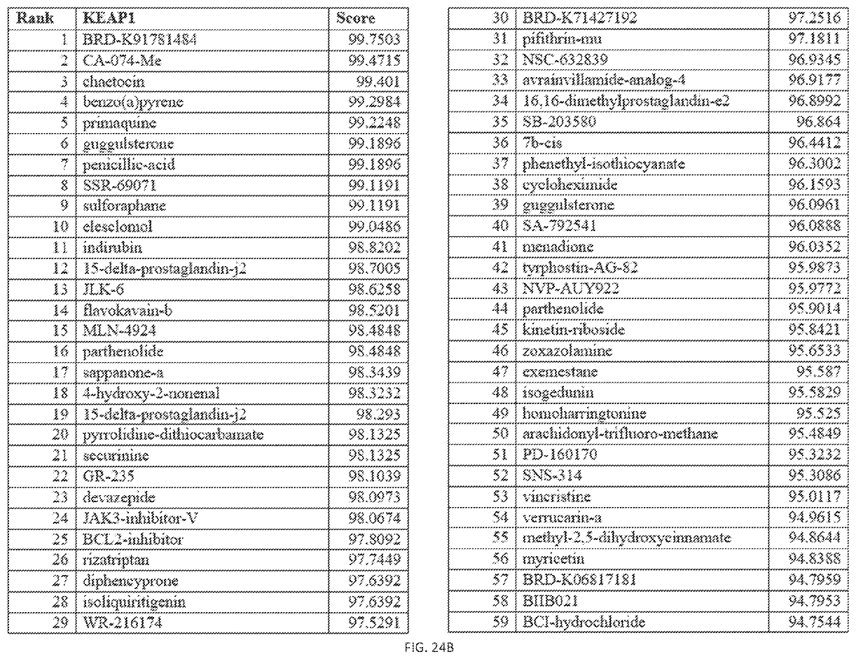

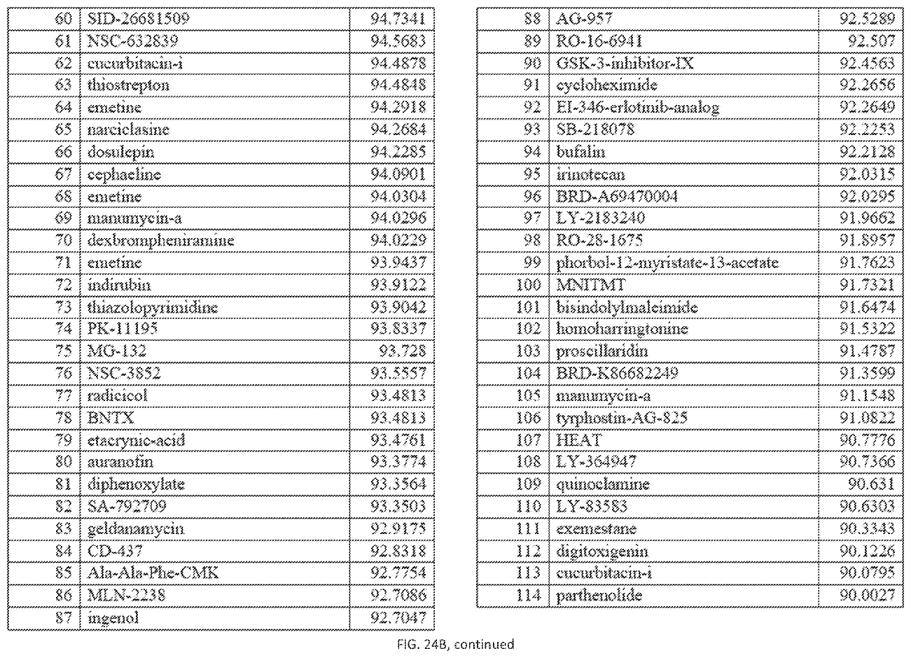

CMAP scores how well each compound's transcriptional profile matches the query signature from −100 to +100, with a score of +100 indicating a complete match. In the Nfe2l2-queried signature, 187 compounds received a Connectivity Score above CMAP's recommended cutoff of +90, whereas 114 compounds scored above +90 in the Keap1-derived signature (FIGS. 4A-4B, 24A and 24B). Among the top 20 candidate compounds prioritized per signature, 7 compounds overlapped between the two queries (FIG. 4C). Exemestane and JQ-1 were two overlapping candidate compounds with high scores in both the Nfe2l2-derived and Keap1-derived signatures. Exemestane is an FDA-approved aromatase inhibitor indicated for the treatment of estrogen-receptor positive breast cancer. JQ-1 is a potent inhibitor of the BET family of bromodomain-containing proteins, displacing them from acetylated lysine residues on histones. BET inhibitors structurally similar to JQ-1 are being considered in clinical trials to treat a variety of cancers.

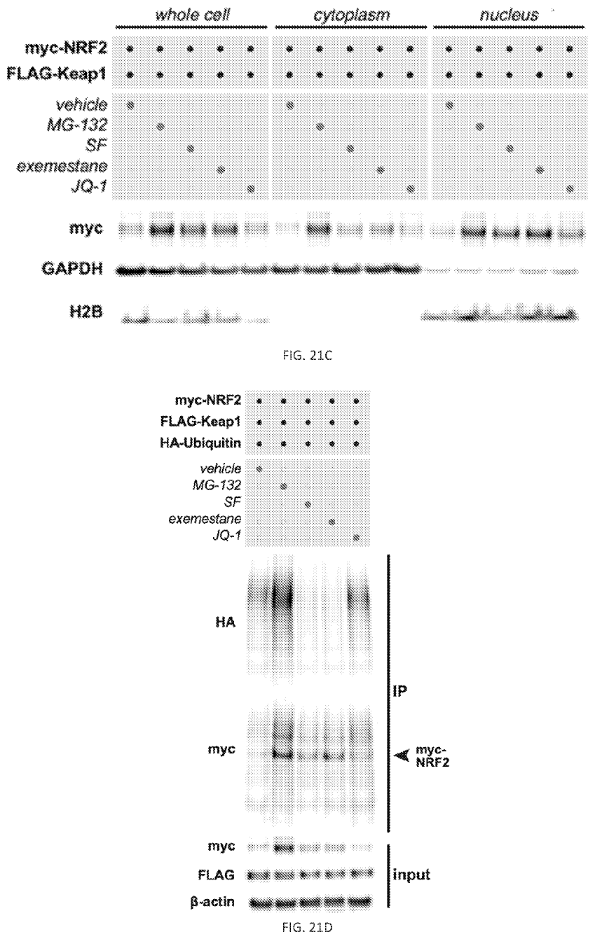

To test whether exemestane or JQ-1 induced the NRF2 transcriptional network as predicted in silico, both compounds were applied to a reporter cell line in which changes in NRF2 activity are coupled to the expression of firefly luciferase. In these cells, the promoter controlling luciferase expression contains several NRF2 binding sites, thereby directly tying luciferase expression to NRF2 activity. Inhibiting KEAP1 with sulforaphane dose-dependently increased luciferase expression in the NRF2 reporter line. Exemestane similarly increased luciferase expression (FIG. 4D) and upregulated the canonical NRF2 target Ngo1, suggesting that exemestane also promoted NRF2 transcriptional activity. In contrast, JQ-1 did not stimulate luciferase expression, ostensibly suggesting that it did not induce the NRF2 transcriptional network (FIG. 4D). However, subsequent re-analysis of RNA-sequencing data uncovered that JQ-1 surprisingly upregulated a number of canonical NRF2 target genes in primary human dermal fibroblasts, such as NQO1, FTL, PRDX1, TXN, and EGR1 (FIG. 4E). JQ-1 also upregulates NQO1 and HMOX1 in corneal fibroblasts and monocytes. Unbiased gene ontology and genetic network analyses detected that the NRF2 pathway was the second most upregulated pathway after treatment with JQ-1 (FIG. 4F). JQ-1 thus potently induced much of the NRF2 transcriptional network, but by a novel mechanism that may not involve NRF2 itself. JQ-1 may upregulate these genes by remodeling chromatin through BET proteins or activating alternative unknown transcription factors, but the exact mechanisms remain presently unclear. Consistent with this, JQ-1 neither stabilizes NRF2 nor inhibits its ubiquitination, whereas both sulforaphane and exemestane do. Sulforaphane and exemestane also both promote nuclear translocation of NRF2, but JQ-1 does not (FIGS. 21A-21D). JQ-1 might instead upregulate antioxidant genes by remodeling chromatin through BET proteins, stimulating NRF2 in an unconventional manner, or activating alternative transcription factors, but the exact mechanisms are presently unclear.

As exemestane and JQ-1 both recruited the NRF2 transcriptome, whether they could lower allodynia in the constrictive mouse model of trigeminal neuralgia was evaluated. Just as with sulforaphane (FIG. 3A), mice treated with either exemestane or JQ-1 were much less sensitive to both crude touch (FIGS. 4G and 4I) and application of ice-cold acetone to the ligated vibrissal pad skin surface (FIGS. 4H and 4J). By immunohistochemistry, exemestane robustly increased NRF2 expression in neurons of the trigeminal ganglion (FIG. 4K). Directly applying exemestane to the maxillary nerve similarly lessened both mechanical and cold allodynia (FIGS. 4L-4M), suggesting that exemestane exerted analgesia locally at the nerve. In assessing myelin basic protein staining, vehicle-treated nerves exhibited fewer myelinated axons after constriction. Treatment with exemestane or JQ-1 resulted in better preserved nerve architecture with increased density of myelinated axons of varying sizes (FIGS. 6C-6D).

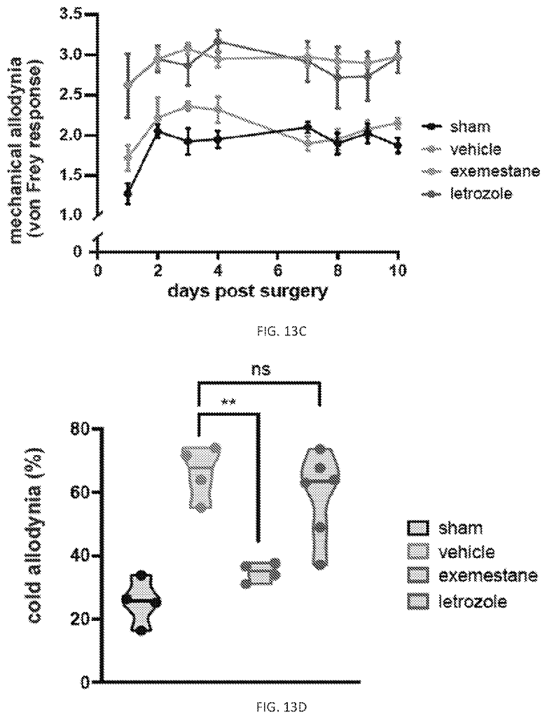

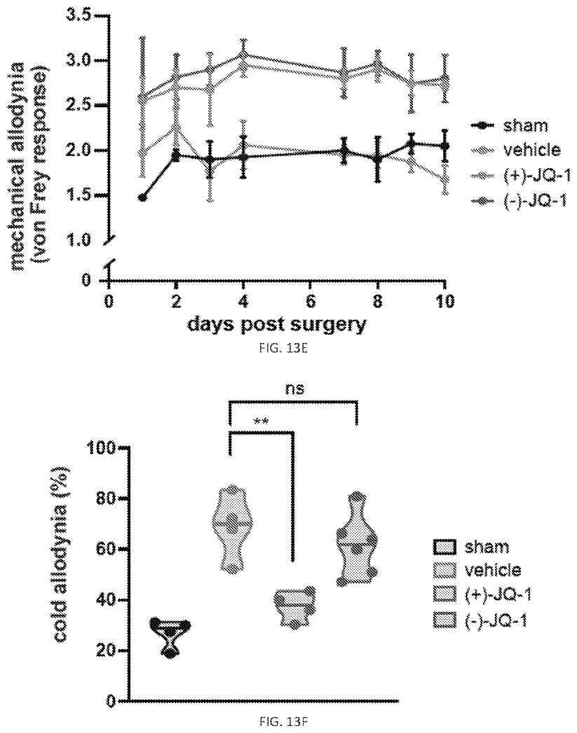

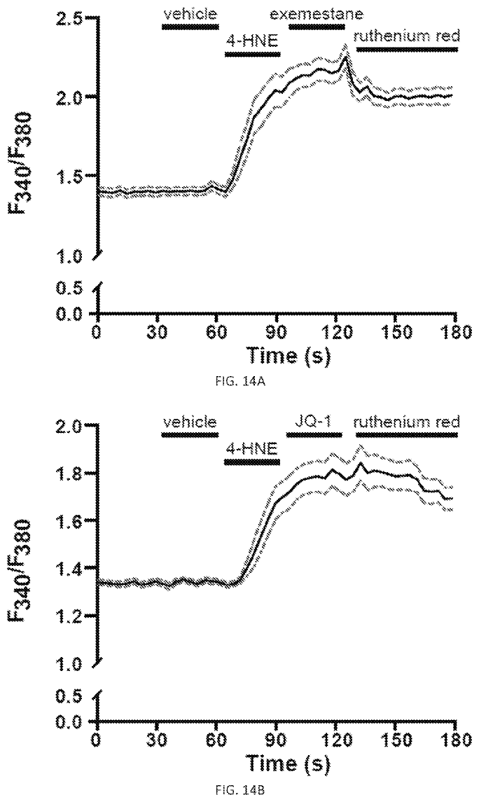

To assess whether exemestane or JQ-1 were acting through alternative off-target mechanisms, nocifensive behavior was also evaluated in mice treated with either letrozole or (−)-JQ-1. Letrozole is a structurally unrelated but potent aromatase inhibitor like exemestane, whereas (−)-JQ-1 is the inactive stereoisomer of (+)-JQ1 (FIGS. 13A-13B). While exemestane and (+)-JQ-1 lowered both mechanical and cold allodynia, neither letrozole nor (−)-JQ-1 did (FIGS. 13C-13F). The analgesic activity of exemestane and JQ-1 are thus less likely due to alternative effects from aromatase inhibition or non-specific changes in DNA topology.

To next test whether sulforaphane, exemestane, and JQ-1's analgesic activities are dependent on NRF2, NRF2^{-/-} mice were treated with all three drugs. Both sulforaphane and exemestane lose their analgesic effects in NRF2^{-/-} mice (FIGS. 22A-22D), suggesting that their mechanisms of action require NRF2. Curiously, JQ-1 still retains some of its analgesic activity. JQ-1 lowers both mechanical and cold allodynia in NRF2^{-/-} mice (FIGS. 22E-22F), though the effects are more muted in comparison to WT mice. Considering that JQ-1 does not biochemically influence NRF2 in the same manner as sulforaphane and exemestane (FIGS. 21A-21D), this further suggests that how JQ-1 upregulates the NRF2 transcriptional network may only partially depend on NRF2 itself.

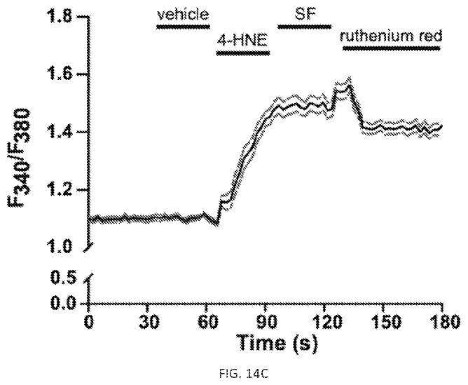

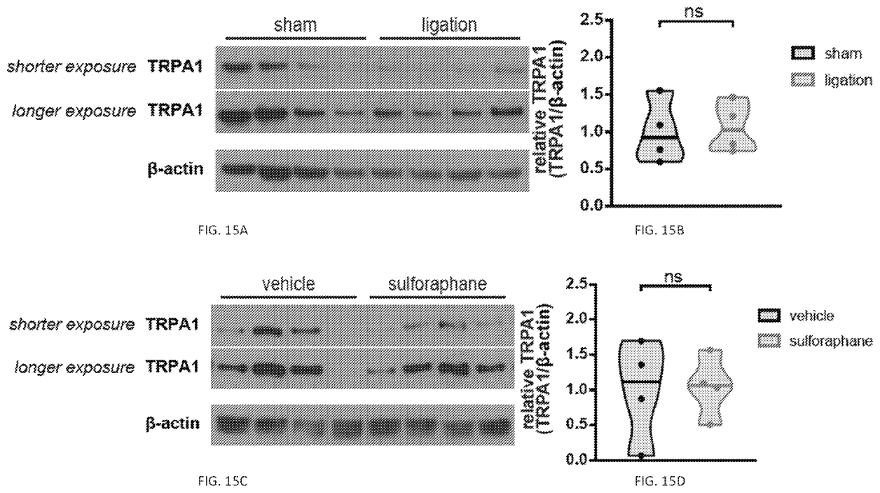

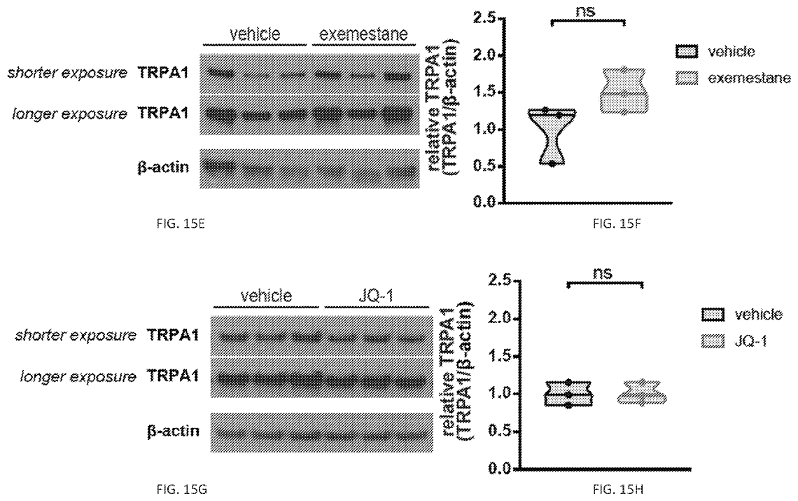

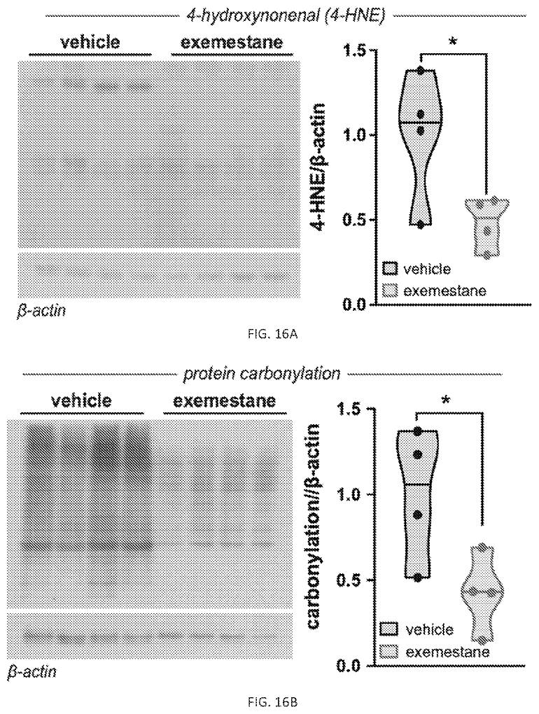

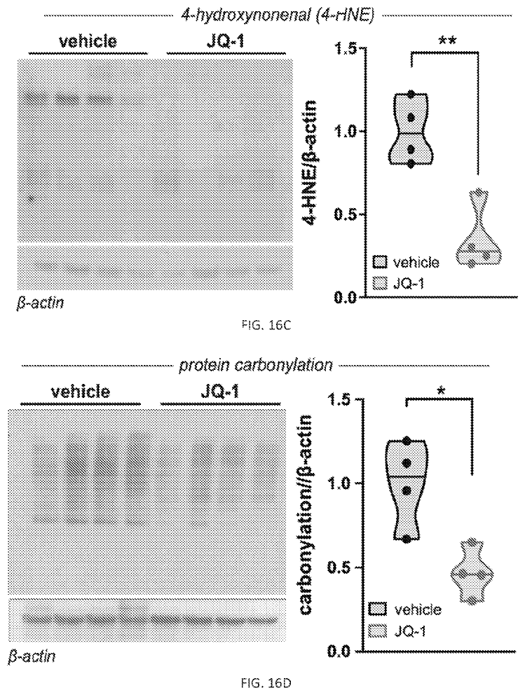

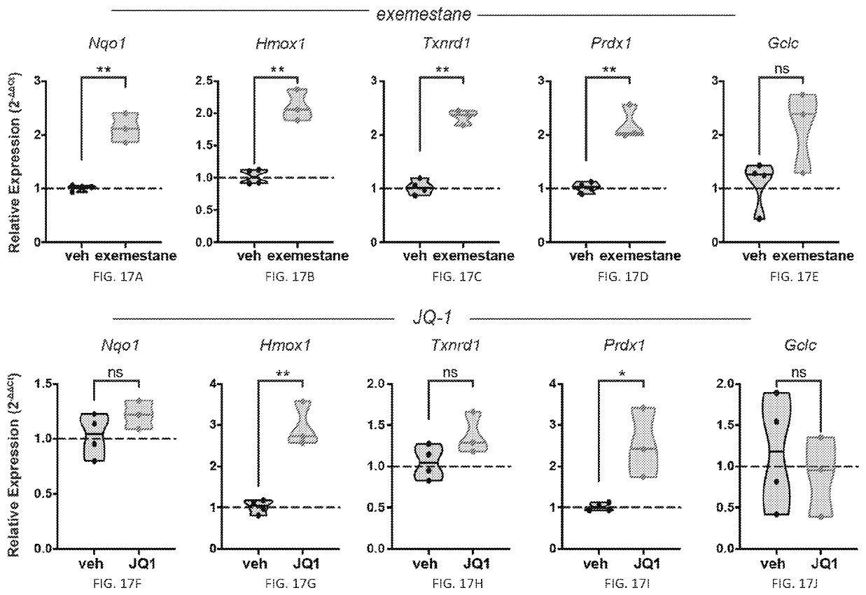

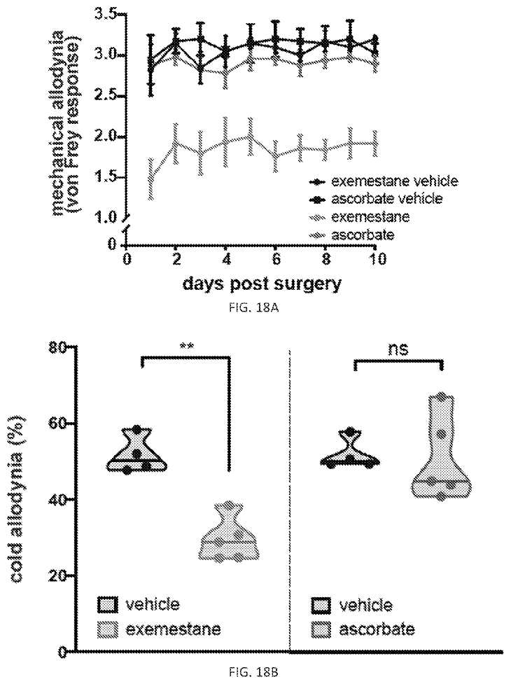

Neither sulforaphane, exemestane, nor JQ-1 inhibited TRPA1 channel activity, suggesting that their analgesic effects were not mediated by directly inhibiting the nociceptor itself (FIGS. 14A-14C). Sulforaphane, exemestane, and JQ-1 did not affect TRPA1 expression either (FIGS. 15A-D). Instead, exemestane or JQ-1 may limit oxidative stress. Like with sulforaphane, pre-treating mice with exemestane or JQ-1 reduced the levels of 4-HNE (FIGS. 16A and 16C) and protein carbonylation (FIGS. 16B and 16D) after nerve ligation. In exploring which genes of the NRF2 transcriptional axis exemestane and JQ-1 may influence, exemestane and JQ-1 were found to differentially upregulate slightly different NRF2 targets and to varying degrees (FIGS. 17A-17J). For example, JQ-1 treatment most potently upregulated Hmox1 followed by Prdx1, but did not increase Txnrd1 expression. Exemestane upregulated Nqo1, Hmox1, Txrnd1, and Prdx1.

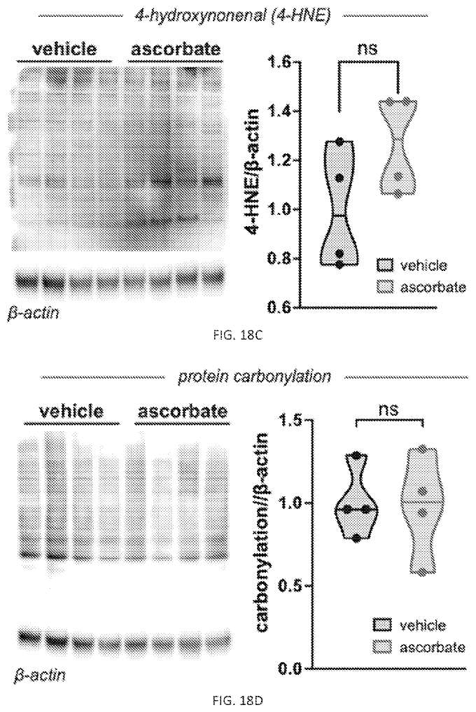

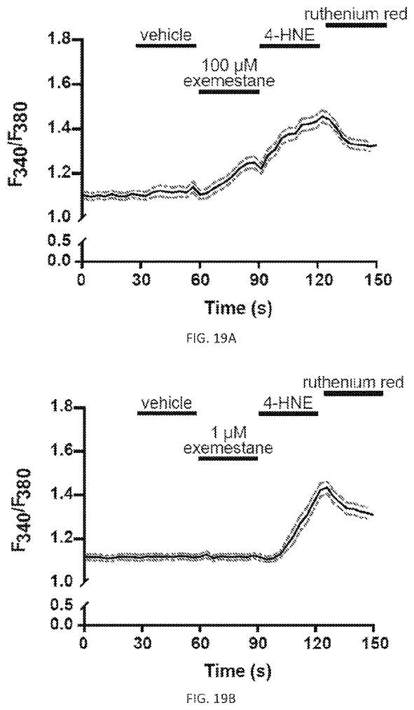

It was considered whether bypassing NRF2 and directly administering an antioxidant might also reduce allodynia. Chronically treating mice with the antioxidant ascorbate did not reduce either mechanical or cold allodynia (FIGS. 18A-18B) or the levels of 4-HNE and protein carbonylation (FIGS. 18C-18D), underscoring the value of specifically targeting the NRF2 antioxidant network in sustaining analgesia. Because most small molecule antioxidants like ascorbate act by stoichiometrically scavenging ROS directly, they are likely most effective when administered very frequently and at high dosages. In addition, contrary to direct administration of an antioxidant, the effects of sulforaphane, exemestane, and JQ-1 persisted 24 hours after treatment.

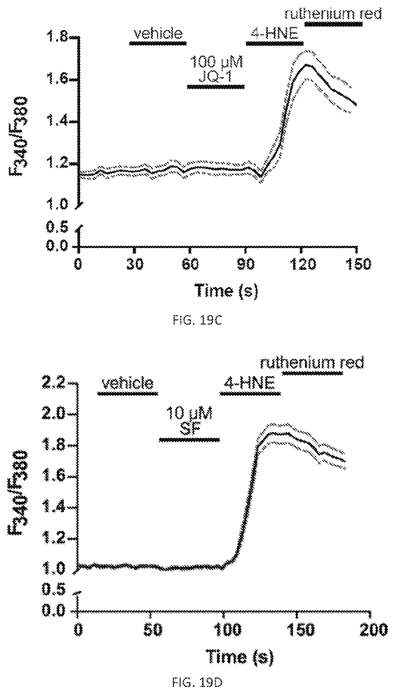

Clinically-relevant concentrations of exemestane (1-100 μm) did not activate TRPA1, though supratherapeutic concentrations might and thus should be avoided when treating patients (FIGS. 19A-19B). Even concentrations of JQ-1 as high as 100 μM do not activate TRPA1 (FIG. 19C). Neither sulforaphane nor JQ-1 activate TRPA1 (FIGS. 19C-19D).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A method of treating or preventing trigeminal nerve pain in a subject comprising administering to the subject a therapeutically or prophylactically effective amount of at least one modulator of oxidative stress or a composition thereof.

2. The method of claim 1, wherein the at least one modulator of oxidative stress comprises a reactive oxygen species suppressant, an activator of anti-oxidative stress genes, or a combination thereof.

3. The method of claim 1 or claim 2, wherein the at least one modulator of oxidation stress comprises an inhibitor of transient receptor potential ankyrin 1 (TRPA1).

4. The method of claim 3, wherein the inhibitor of TRPA1 is selected from the group consisting of ruthenium red, AM-0902, and combinations thereof.

5. The method of any of claims 1-4, wherein the at least one modulator of oxidative stress comprises a nuclear factor erythroid 2-related factor 2 (NRF2) transcription network activator.

6. The method of claim 5, wherein the NRF2 transcription network activator is selected from the group consisting of sulforaphane, exemestane, JQ-1, and combinations thereof.

7. The method of any of claim 1-6, wherein the administering comprises perineural injection, systemic injection, or intranasal administration.

8. The method of any of claims 1-7, wherein the administering comprises administration to at least one trigeminal nerve branch.

9. The method of any of claims 1-8, wherein the administering comprises administration to sinus, inferior two-thirds of nasal cavity, or nasal septum.

10. The method of any of claims 1-9, wherein the trigeminal nerve pain comprises trigeminal neuralgia or trigeminal neuropathy.

11. Use of at least one modulator of oxidative stress or a composition thereof for treating or preventing trigeminal nerve pain in a subject comprising administering to the subject a therapeutically or prophylactically effective amount of at least one modulator of oxidative stress or a composition thereof.

12. The use of claim 11, wherein the at least one modulator of oxidative stress comprises a reactive oxygen species suppressant, an activator of anti-oxidative stress genes, or a combination thereof.

13. The use of claim 11 or claim 12, wherein the at least one modulator of oxidation stress comprises an inhibitor of transient receptor potential ankyrin 1 (TRPA1).

14. The use of claim 13, wherein the inhibitor of TRPA1 is selected from the group consisting of ruthenium red, AM-0902, and combinations thereof.

15. The use of any of claims 11-14, wherein the at least one modulator of oxidative stress comprises a nuclear factor erythroid 2-related factor 2 (NRF2) transcription network activator.

16. The use of claim 15, wherein the NRF2 transcription network activator is selected from the group consisting of sulforaphane, exemestane, JQ-1, and combinations thereof.

17. The use of any of claim 11-16, wherein the administering comprises perineural injection, systemic injection, or intranasal administration.

18. The use of any of claims 11-17, wherein the administering comprises administration to at least one trigeminal nerve branch.

19. The use of any of claims 11-18, wherein the administering comprises administration to sinus, inferior two-thirds of nasal cavity, or nasal septum.

20. The use of any of claims 11-19, wherein the trigeminal nerve pain comprises trigeminal neuralgia or trigeminal neuropathy.
